(19) United States
(12) Patent Application Publication
(10) Pub. No.: US 2025/0368984 A1
(43) Pub. Date: Dec. 4, 2025

(54) DISCOVERY OF PACLITAXEL AND CARBOPLATIN SENSITIVITY GENES VIA CRISPR SCREEN AND METHYLATION IN GLIOBLASTOMA PATIENTS

(71) Applicant: Northwestern University, Evanston, IL (US)

(72) Inventors: Mark William Youngblood, Evanston, IL (US); Crismita Clement Dmello, Evanston, IL (US); Adam Mendel Sonabend Worthalter, Evanston, IL (US); Roger Stupp, Evanston, IL (US)

(73) Assignee: Northwestern University, Evanston, IL (US)

(21) Appl. No.: 19/223,735

(22) Filed: May 30, 2025

Related U.S. Application Data
(60) Provisional application No. 63/654,255, filed on May 31, 2024.

(51) Int. Cl. C12N 15/10; G16B 35/20

(52) U.S. Cl. CPC C12N 15/1089; G16B 35/20

(57) ABSTRACT

Provided is a method to detect cancer biomarkers, wherein the biomarkers indicate whether a patient will be susceptible to a chemotherapeutic agent.

### CROSS-REFERENCE TO RELATED APPLICATIONS

This application claims priority to U.S. Provisional Application No. 63/654,255, filed May 31, 2024, the entire contents of which are incorporated herein by reference.

### BACKGROUND

Despite decades of intensive research in glioblastoma chemotherapy, predicting which patients will respond to specific medications remains a challenge. There is a need for improved methods to predict which patients will respond to specific medications. This includes the need to develop predictive biomarkers in cancer tissue, in particular in gliomas and glioblastoma.

### SUMMARY

Glioblastoma is a deadly form of brain cancer in need of novel therapeutic approaches to improve outcomes. Response to chemotherapy is heterogeneous, and likely depends on patient-specific molecular features that confer relative sensitivity or resistance to specific agents. As described herein, Applicant used genome-wide CRISPR screens to identify genes associated with sensitivity to the chemotherapies paclitaxel, carboplatin, or combined treatment. Among the resulting candidate biomarkers, Applicant correlates overall survival with DNA methylation levels, using recent clinical trials in which glioblastoma patients were treated with these drugs.

As a result, the approach described herein identifies unbiased biomarkers of sensitivity to paclitaxel and carboplatin that play a functional role in cellular response to these medications that can be interrogated using already available and Clinical Laboratory Improvement Amendments (CLIA) certified DNA methylation platforms. The fact that these biomarkers predict survival for glioma patients treated with these drugs ensures reproducibility and efficacy in human patients.

Applicant's sequential approach of (i) genome-wide CRISPR screen (to identify functional mediators of drug response) followed by (ii) DNA methylation validation using patient samples maximize chances that the resulting biomarkers will be clinically useful. No previous methods have used the combination approach of CRISPR and DNA methylation to identify paclitaxel and carboplatin sensitivity in gliomas, including glioblastoma.

### FIGURES

FIG. 1A-1B shows the CRISPR screen and cell numbers after exposure of in GBM6 cells to paclitaxel, carboplatin, or both. FIG. 1A shows the chemotherapeutic agents at high drug concentrations. FIG. 1B shoes the chemotherapeutic agents at low concentrations.

FIG. 2 shows candidate biomarker genes identified in the full-genome CRISPR screen of Dmello et al 2022 (doi.org/10.1158/1078-0432.CCR-21-2563). In this screen, H4 glioma cells were exposed to paclitaxel and enriched genes (i.e. candidate biomarkers) were identified using DEseq and sgRSEA analysis. These genes are expected to confer sensitivity to paclitaxel (i.e. their knock-down results in resistance to the drug) and exhibit higher fold-change (left panel) and sgRSEA enrichment score (right panel) compared to DMSO control.

FIGS. 3A-3D. Enrichment of Sensitivity Genes in Carboplatin, Paclitaxel, and Combination CRISPR Screens. (FIG. 3A) The GBM6 cell line was modified using a CRISPR screening library that generates a whole genome knockout wherein each cell harbors a single gene knock-out. After treatment of each clonal population with carboplatin (vs. control), the relative growth advantage conferred by each gene knock-out is measured. The extent of growth advantage is plotted as the Beta score (relative enrichment of gene sgRNAs in carboplatin vs. control). Genes conferring the highest sensitivity to carboplatin are labeled and colored in red. Similar plots are shown for the (FIG. 3B) paclitaxel and (FIG. 3C) combination CRISPR screens, with the associated sensitivity genes also highlighted. (FIG. 3D) Overlap in sensitivity genes between the three CRISPR screens (high concentrations of chemotherapeutic agent) is shown. Only a single gene was found to confer sensitivity in all three conditions.

FIGS. 4A-4B. Longitudinal Measurement of MRI Enhancement. Patients underwent repeated opening of the blood-brain barrier during outpatient clinic visits (‘cycles’) in which paclitaxel was administered. Periodic MRI scans were obtained during treatment to measure changes in enhancement (tumor growth). (FIG. 4A) Example longitudinal MRI results are shown from a patient with delayed tumor growth (Patient 110) and early recurrence (Patient 109). (FIG. 4B) Quantification of enhancement was performed at each timepoint among the entire brain, as well as within the region of blood-brain barrier opening. These data can be correlated with DNA methylation levels of individual genes to determine biomarker efficacy.

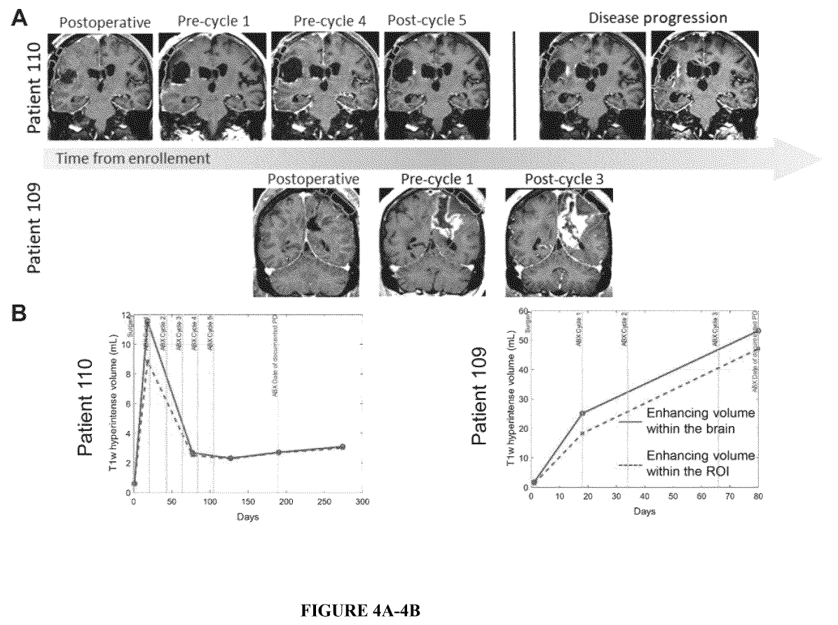

FIGS. 5A-5C. Relationship of DNA Methylation Level with Change in MRI Enhancement for an Example Probe. (FIG. 5A) The level of DNA methylation (M-value) (y-axis) of probe cg03052784 (IL1R1 gene) is plotted against the change in MRI enhancement levels from the beginning to end of the Sonocloud trial. The two variables are correlated. Il1R1 was identified in the paclitaxel CRISPR screen as a sensitivity conferring gene (see Table 1) FIG. 5B) Axial images are shown from contrast-enhanced MRI scans at the beginning and end of the Sonocloud trial for an example patient, circled in green in FIG. 5A). The difference in enhancement from these two time points is used to calculate the x-axis values in FIG. 7A. (FIG. 5C) A similar example is shown for a patient with less change in enhancement, circled in pink in FIG. 5A.

FIG. 6. Correlation of DNA Methylation Probes with Gene Expression. Using paired RNA-seq and DNA methylation data from 117 glioblastoma patients in The Cancer Genome Atlas, correlation was performed between each methylation probe and expression of the nearest gene. The significance of these associations was normally distributed, and did not vary based on location of a probe in a promoter region (left panel vs. right panel), or relative position to a CpG loci (dot color). These results identified which DNA methylation probes affect gene expression levels in GBM patients, and thereby could phenocopy the CRISPR screen.

FIG. 7. Association of DNA Methylation Probes with GBM Patient Survival. DNA methylation data from The Cancer Genome Atlas was used to determine each probes association with overall survival (FIG. 7A) and progression free (radiographic) survival

(FIG. 7B). Only probes that overlapped with the 51 genes identified during the paclitaxel CRISPR sensitivity screen (see Table 1) were considered. After correction for multiple-hypothesis testing, no probes were found to be significantly correlated with overall survival or progression free survival. This confirms that methylation levels of these genes are not prognostic of GBM response in the absence of paclitaxel treatment.

FIG. 8A-8B shows TMEM131 (probe cg16445423) correlation data with gene expression and overall survival. (FIG. 8A) The level of DNA methylation (y-axis) of probe cg16445423 was correlated with expression of nearby gene TMEM131 (x-axis), which was previously identified as a sensitivity conferring gene in the paclitaxel CRISPR screen (sec Table 1). Lower levels of methylation at this probe result in higher levels of gene expression. (FIG. 8B) Patients with decreased DNA methylation of cg16445423 (higher expression) exhibited longer overall survival after treatment with paclitaxel during BBB opening.

FIG. 9A-9C. Associations of an Example DNA Methylation Probe with Patient Outcome after Paclitaxel Treatment (FLT3). (FIG. 9A) The level of DNA methylation (y-axis) of probe cg04387836 was strongly associated with overall survival after treatment with Paclitaxel during BBB opening. Patients with lower methylation exhibited longer survival. (FIG. 9B) This probe is located within the gene FLT3, a type of tyrosine kinase receptor, which was identified as conferring paclitaxel sensitivity in the CRISPR screen (sec Table 1). (FIG. 9C) A Kaplan-Meier plot shows the difference in survival time among patients with high (top 50%) vs low (bottom 50%) methylation levels of cg04387836 after treatment with Paclitaxel during BBB opening.

FIG. 10 shows a poorly correlated probe cg13483502 (HMCN2). HMCN2 was identified as conferring paclitaxel sensitivity in the CRISPR screen (see Table 1) There is no linear relationship associated with either change in MRI enhancement or overall survival.

### DETAILED DESCRIPTION

It is to be appreciated that certain aspects, modes, embodiments, variations and features of the present methods are described below in various levels of detail in order to provide a substantial understanding of the present technology.

The present disclosure is not to be limited in terms of the particular embodiments described in this application, which are intended as single illustrations of individual aspects of the disclosure. All the various embodiments of the present disclosure will not be described herein. Many modifications and variations of the disclosure can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled.

In practicing the present technologies, many conventional techniques in molecular biology, protein biochemistry, cell biology, microbiology and recombinant DNA are used. Sec, e.g., Sambrook and Russell eds. (2001) Molecular Cloning: A Laboratory Manual, 3^{rd} edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

Glioblastoma (GBM) remains an incurable disease requiring new treatments and drug delivery modalities. The dire clinical outcomes in GBM patients are explained, at least in part, by the blood-brain barrier (BBB) which can prevent achieving effective concentrations of, for example, systemically administered chemotherapies, antibody-based immunotherapies, and targeted therapies into the brain (Banks, W.A., Nat Rev Drug Discov 15, 275-292 (2016)). Further, the glioblastoma response to chemotherapy is heterogeneous, and likely depends on patient-specific molecular features that confer relative sensitivity or resistance to specific agents. Therefore, it is important to identify the glioblastoma which are responsive to chemotherapy agents such as paclitaxel and/or carboplatin prior to delivery of the agent across the blood brain barrier. The technologies described herein be applied to glioblastomas and other cancers.

### Definitions

Unless defined otherwise, all technical and scientific terms used herein have the meaning commonly understood by a person skilled in the art to which this disclosure belongs. The following references provide one of skill with a general definition of many of the terms used in the present disclosure. Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise. The terminology used herein is for the purpose of describing particular embodiments only and is not intended to be limiting of the disclosure.

As used herein, the single forms “a”, “an”, and “the” are intended to include the plural forms as well, unless the context clearly indicates otherwise.

Unless the context indicates otherwise, it is specifically intended that the various features of the invention described herein can be used in any combination. Moreover, the disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

As used here, the term “about,” when used to modify a numerical value, indicates that deviations of up to 10% above and below the numerical value, including the numerical value, remain within the intended meaning of the recited value. For example, “about 10” should be understood as both “10” and “9-11”.

As used herein, the term “administering” of an agent to a subject includes any route of introducing or delivering the agent to the subject to perform its intended function. Administration can be carried out by any suitable route, including, but not limited to, intravenously, intramuscularly, intraperitoneally, subcutaneously, and other suitable routes as described herein. Administration includes self-administration and the administration by another.

An “effective amount” or “efficacious amount” refers to the amount of an agent, or combined amounts of two or more agents, that, when administered for the treatment of a mammal or other subject, is sufficient to effect such treatment for the disease. The “effective amount” will vary depending on the agent(s), the disease and its severity and the age, weight, etc., of the subject to be treated. The term “effective amount” refers to a quantity sufficient to achieve a desired effect. In the context of therapeutic or prophylactic applications, the effective amount will depend on the type and severity of the condition at issue and the characteristics of the individual subject, such as general health, age, sex, body weight, and tolerance to pharmaceutical compositions. With respect to immunogenic compositions, in some embodiments the effective amount will depend on the intended use, the degree of immunogenicity of a particular antigenic compound, and the health/responsiveness of the subject's immune system, in addition to the factors described above. The skilled artisan will be able to determine appropriate amounts depending on these and other factors.

As used herein, the term “combination therapy” refers to those situations in which two or more different pharmaceutical agents are administered in overlapping regimens so that the subject is simultaneously exposed to both agents. When used in combination therapy, two or more different agents may be administered simultaneously or separately. This administration in combination can include simultaneous administration of the two or more agents in the same dosage form, simultaneous administration in separate dosage forms, and separate administration. That is, two or more agents can be formulated together in the same dosage form and administered simultaneously. Alternatively, two or more agents can be simultaneously administered, wherein the agents are present in separate formulations. In another alternative, a first agent can be administered just followed by one or more additional agents. In the separate administration protocol, two or more agents may be administered a few minutes apart, or a few hours apart, or a few days apart.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of”' shall mean excluding more than trace elements of other ingredients for claimed compositions and substantial method steps. Embodiments defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

As used herein, the term “effective amount” or “therapeutically effective amount” refers to a quantity of an agent sufficient to achieve a beneficial or desired clinical result upon treatment. In the context of therapeutic applications, the amount of a therapeutic agent administered to the subject can depend on the type and severity of the disease or condition and on the characteristics of the individual, such as general health, age, sex, body weight, effective concentration of the therapeutic agent administered, and tolerance to drugs. It can also depend on the degree, severity, and type of disease. The skilled artisan will be able to determine appropriate dosages depending on these and other factors. An effective amount can be administered to a subject in one or more doses. In terms of treatment, an effective amount is an amount that is sufficient to palliate, ameliorate, stabilize, reverse or slow the progression of the disease, or otherwise reduce the pathological consequences of the disease. The effective amount is generally determined by the physician on a case-by-case basis and is within the skill of one in the art.

As used herein, the term “reduce” or “decrease” means to alter negatively by at least about 5% including, but not limited to, alter negatively by about 5%, by about 10%, by about 25%, by about 30%, by about 50%, by about 75%, or by about 100%.

In certain embodiments, the terms “disease” “disorder” and “condition” are used interchangeably herein, referring to a cancer, a status of being diagnosed with a cancer, or a status of being suspect of having a cancer.

As used herein, a “cancer” is a disease state characterized by the presence in a subject of cells demonstrating abnormal uncontrolled replication and may be used interchangeably with the term “tumor.” In some embodiments, the cancer is a glioma or glioblastoma. “Cell associated with the cancer” refers to those subject cells that demonstrate abnormal uncontrolled replication.

“Cancer”, which is also referred to herein as “tumor”, is a known medically as an uncontrolled division of abnormal cells in a part of the body, benign or malignant. In one embodiment, cancer refers to a malignant neoplasm, a broad group of diseases involving unregulated cell division and growth, and invasion to nearby parts of the body. Non-limiting examples of cancers include carcinomas, sarcomas, leukemia and lymphoma, e.g., colon cancer, colorectal cancer, rectal cancer, gastric cancer, esophageal cancer, head and neck cancer, breast cancer, brain cancer, lung cancer, stomach cancer, liver cancer, gall bladder cancer, or pancreatic cancer. In one embodiment the brain cancer is a glioma, for example a glioblastoma. In one embodiment, the term “cancer” refers to a solid tumor, which is an abnormal mass of tissue that usually does not contain cysts or liquid areas, including but not limited to, sarcomas, carcinomas, and certain lymphomas (such as Non-Hodgkin's lymphoma). In another embodiment, the term “cancer” refers to a liquid cancer, which is a cancer presenting in body fluids (such as, the blood and bone marrow), for example, leukemias (cancers of the blood) and certain lymphomas.

Additionally or alternatively, a cancer may refer to a local cancer (which is an invasive malignant cancer confined entirely to the organ or tissue where the cancer began), a metastatic cancer (referring to a cancer that spreads from its site of origin to another part of the body), a non-metastatic cancer, a primary cancer (a term used describing an initial cancer a subject experiences), a secondary cancer (referring to a metastasis from primary cancer or second cancer unrelated to the original cancer), an advanced cancer, an unresectable cancer, or a recurrent cancer. As used herein, an advanced cancer refers to a cancer that had progressed after receiving one or more of: the first line therapy, the second line therapy, or the third line therapy.

A “solid tumor” is an abnormal mass of tissue that usually does not contain cysts or liquid areas. Solid tumors can be benign or malignant. Different types of solid tumors are named for the type of cells that form them. Examples of solid tumors include sarcomas, carcinomas, gliomas, and lymphomas. The solid tumor can be localized or metastatic.

CRISPR (Clustered Regularly Interspaced Short Palindromic Repeats) is an acronym for DNA loci that contain multiple, short, direct repetitions of base sequences. The prokaryotic CRISPR/Cas system has been adapted for use as gene editing (silencing, enhancing or changing specific genes) for use in eukaryotes (see, for example, Cong, Science, 15:339(6121):819-823 (2013) and Jinek, et al., Science, 337(6096):816-21 (2012)). By transfecting a cell with elements including a Cas gene and specifically designed CRISPRs, nucleic acid sequences can be cut and modified at any desired location. Methods of preparing compositions for use in genome editing using the CRISPR/Cas systems are described in detail in US Pub. No. 2016/0340661, US Pub. No. 20160340662, US Pub. No. 2016/0354487, US Pub. No. 2016/0355796, US Pub. No. 20160355797, and WO 2014/018423, which are specifically incorporated by reference herein in their entireties.

Thus, as used herein, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer”, “guide RNA” or “gRNA” in the context of an endogenous CRISPR system), or other sequences and transcripts from a CRISPR locus. One or more tracr mate sequences operably linked to a guide sequence (e.g., direct repeat-spacer-direct repeat) can also be referred to as “pre-crRNA” (pre-CRISPR RNA) before processing or crRNA after processing by a nuclease.

In some embodiments, one or more vectors driving expression of one or more elements of a CRISPR system are introduced into a target cell such that expression of the elements of the CRISPR system direct formation of a CRISPR complex at one or more target sites. While the specifics can be varied in different engineered CRISPR systems, the overall methodology is similar. A practitioner interested in using CRISPR technology to target a DNA sequence can insert a short DNA fragment containing the target sequence into a guide RNA expression plasmid. The sgRNA expression plasmid contains the target sequence (about 20 nucleotides), a form of the tracrRNA sequence (the scaffold) as well as a suitable promoter and necessary elements for proper processing in eukaryotic cells. Such vectors are commercially available (see, for example, Addgene). Many of the systems rely on custom, complementary oligos that are annealed to form a double stranded DNA and then cloned into the sgRNA expression plasmid. Co-expression of the sgRNA and the appropriate Cas enzyme from the same or separate plasmids in transfected cells results in a single or double strand break (depending of the activity of the Cas enzyme) at the desired target site.

### Methods of Determining Biomarkers

As used herein, the term “biomarker” refers to a characteristic that is objectively measured and evaluated as an indicator of normal biological processes, pathogenic processes, or pharmacological responses to a therapeutic intervention. By way of example but not by way of limitation, biomarkers disclosed herein include regulators of gene expression such as DNA methylation levels. In some embodiments, an aberrant level of DNA methylation (e.g., an increased (enhanced) or decreased (lower) level of methylation at a specific genomic locus in a tumor sample from a subject suffering from cancer) as compared to a control, threshold, or baseline level of DNA methylation is indicative of susceptibility or resistance of the cancer to one or more therapeutic treatments. In some embodiments, the level of DNA methylation as compared to a control, threshold, or baseline level is indicative of susceptibility or resistance of the cancer to one or more therapeutic treatments.

As used herein, “susceptible” and “sensitive” in the context of tumor response to chemotherapeutic agent are used interchangeably.

The biomarkers disclosed herein are genomic locations in which changes in DNA methylation level are predictive of sensitivity to a chemotherapeutic agent. These genomic locations correspond to biomarker genes. In some embodiments, the chemotherapeutic agent is paclitaxel, carboplatin, or a combination of the two.

As used herein, the biomarkers may be referred to as biomarker genes or cancer biomarker genes, which refers to the genomic location (i.e. gene) in which the changes in DNA methylation level are predictive of sensitivity to a chemotherapeutic agent. As described herein, a broader list of candidate genes may be identified by CRISPR screens, and narrowed down to the biomarker genes by DNA methylation analysis.

### Methods of Determining Biomarkers

According to one embodiment, described herein is a method for cancer biomarker identification. The method includes obtaining genome-wide CRISPR screen data for cancer cells exposed to at least one chemotherapeutic agent and determining which genes from the CRISPR screen play a role in susceptibility to the at least one chemotherapeutic agent.

In one embodiment, the CRISPR screen is performed with the Human CRISPR Knockout Pooled (Brunello) sgRNA library. The Brunello sgRNA library is available for purchase, for example at addgene.org. The Brunello sgRNA library includes sgRNAs to make edits (knockdown) over 19,000 genes in the human genome. In other aspects, other sgRNAs are used in the genome-wide CRISPR screen. As used herein, the terms gRNA and sgRNA are used interchangeably.

In one embodiment, the CRISPR screen is a pooled CRISPR screen, wherein the gRNAs are pooled and added to one population of cells. In another embodiment, the CRISPR screen is an arrayed CRISPR screen, wherein a gRNA is added individually to a pool of cells. The specifics of pooled and arrayed CRISPR screens will be known to those skilled in the art. See. e.g. Bock et al (2022) (doi.org/10.1038/s43586-021-00093-4) for additional information on genome wide CRISPR screens.

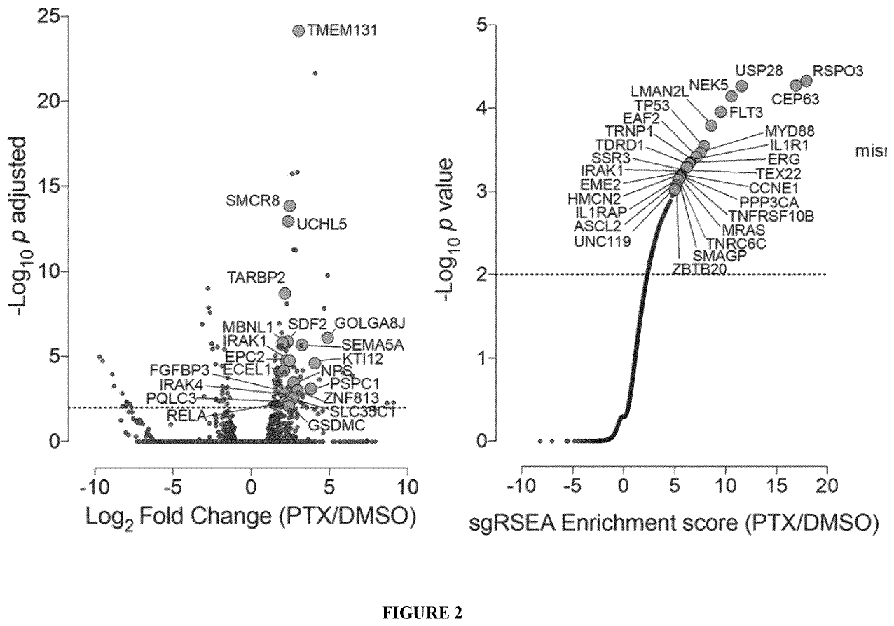

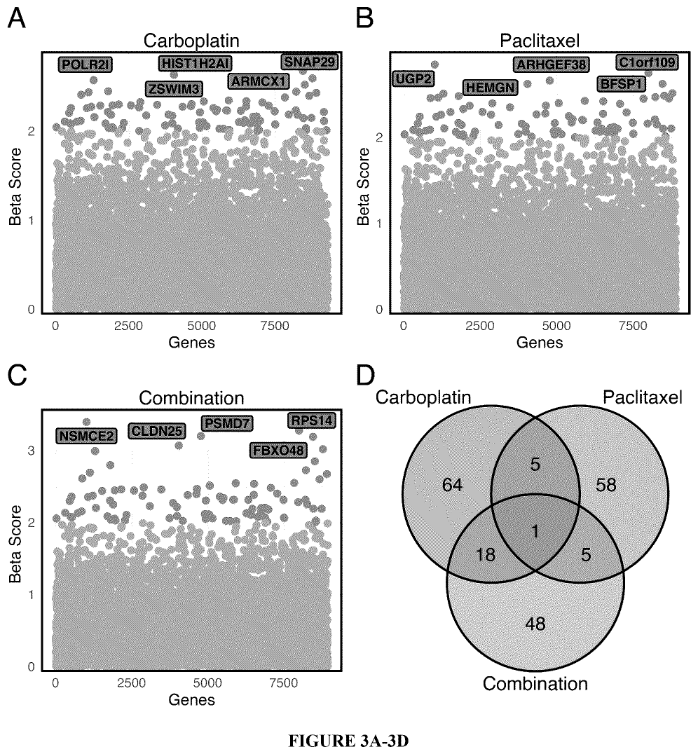

In a pooled CRISPR screen, gRNAs are extracted, sequenced, and enriched gRNAs are identified. To determine enrichment, the gRNAs in the presence of the chemotherapeutic agent are compared to the gRNAs in a negative control. The enriched gRNAs can be corresponded with a gene. Without wishing to be bound by any particular theory, in the chemotherapeutic agent CRISPR screens, genes with enrichment in resistant cells are thought to play a role in conferring chemotherapeutic agent sensitivity. While the definition of “enriched” can be user defined, FIG. 2 shows that the candidate genes identified by Applicant (orange dots) in a paclitaxel screen in H4 cells were at least one log₂ fold higher in paclitaxel exposed cells compared to the control (DEseq algorithm) or have a sgRSEA Enrichment score of approximately 5 or higher in paclitaxel exposed cells compared to the control (sgRSEA algorithm). Similarly, FIG. 3 shows that candidate genes are enriched with a Beta-value greater than approximately 2 (red dots) in the relative enrichment of sgRNAs in GBM6 cells as compared to the control.

Alternatively, in an arrayed CRISPR screen, the susceptibility of individual CRISPR mutants is looked at. In an arrayed chemotherapeutic agent CRISPR screen, mutants which are not susceptible to a chemotherapeutic agent likely have a gene knocked out which plays a role in conferring chemotherapeutic agent sensitivity.

In general, the biomarker genes are specific to the chemotherapeutic agent or combination of chemotherapeutic agents used (FIG. 3D). In a CRISPR screen with paclitaxel, carboplatin, or both, Applicants saw that a majority of biomarker candidate genes were specific to the treatment.

The method further includes identifying and quantifying DNA methylation of probes associated with genes identified in the CRISPR screen in cancer samples collected from a subject with a cancer and treated with the at least one chemotherapeutic agent and quantifying a cancer-progression metric of interest in the subject. DNA methylation is known in the art. Examples of a DNA methylation array include the Illumina EPIC 850K array, and the Infinium MethylationEPIC v2.0 BeadChip (illumina.com/products/by-type/microarray-kits/infinium-methylation-epic.html). DNA methylation arrays can include hundreds of thousands of probes. The DNA methylation levels can be given a Beta-value and/or a M-value. Methods and calculations for determining the methylation levels are known to those in the art. See e.g. Du et al. (2010) (doi.org/10.1186/1471-2105-11-587).

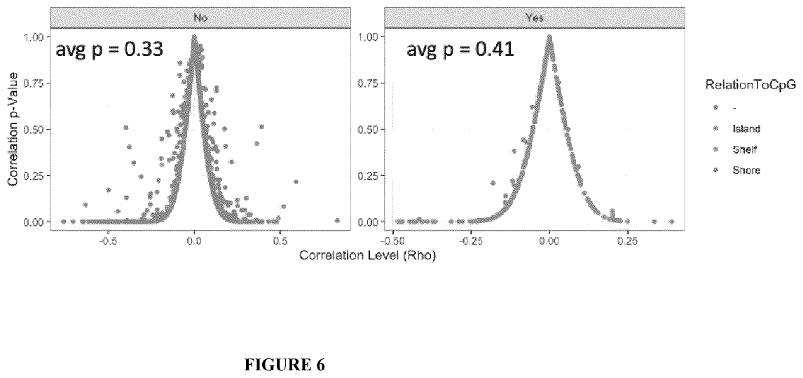

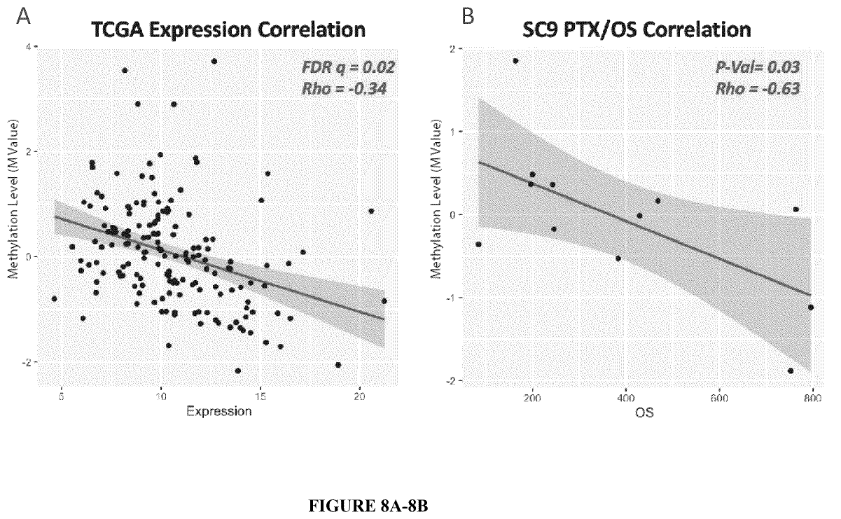

As shown in FIG. 6, DNA methylation correlates with gene expression. In a comparison of RNA-seq data and DNA methylation data from 117 glioblastoma patients, there was a correlation between the DNA methylation probes and gene expression. As shown in FIG. 8, methylation of probe cg16445423 (TMEM131 gene) (M value) was correlated with both gene expression and overall survival of cancer patients treated with paclitaxel.

The method further includes correlating the DNA methylation data with the cancer-progression metric. In a candidate biomarker, the DNA methylation level and cancer-progression metric may be correlated.

The correlation between DNA methylation and cancer-progression metric may be positive or negative, depending on the cancer-progression metric of interest and DNA methylation value of the specific gene. In one aspect, the biomarker indicates susceptibility to a chemotherapeutic agent.

In one embodiment, the cancer cells used in the CRISPR screen are the same or different cancer type as the cancer sample collected from the subject. In one embodiment, the cancer cells used in the CRISPR screen are a glioma or glioblastoma cell line selected from GBM6, A172, SW1088, U118-MG, U87-MG, AM38, KS1, 8 MG-BA, H4, and GB1. In another embodiment the cancer cells used in the CRISPR screen are a breast cancer cell line selected from MDA-MB-231, BT459, Hs578T, and HCC1937 In another embodiment, the cancer cells used in the CRISPR screen are a cell line isolated from a different cancer. In one embodiment the cancer cell lines used in the CRISPR screen are derived from selected from tumors in the brain, breast, ovary, or gastrointestinal tract.

In one embodiment, the subject has breast cancer, ovarian cancer, Kaposi's sarcoma, or glioblastoma.

In one embodiment the cells used in the CRISPR screen are H4 glioma cells and the subject has a glioblastoma. In one embodiment the cells used in the CRISPR screen are GBM6 glioblastoma cells and the subject has a glioblastoma.

In one embodiment, the chemotherapeutic agent used in the CRISPR screen and to treat the subject is selected from bleomycin, cyclophosphamide, doxorubicin, epirubicin, etoposide, 5-fluorouracil, methotrexate, oxaliplatin, temozolomide, cisplatin, paclitaxel and/or carboplatin.

In one embodiment, the chemotherapeutic agent used in the CRISPR screen and to treat the subject is paclitaxel and/or carboplatin.

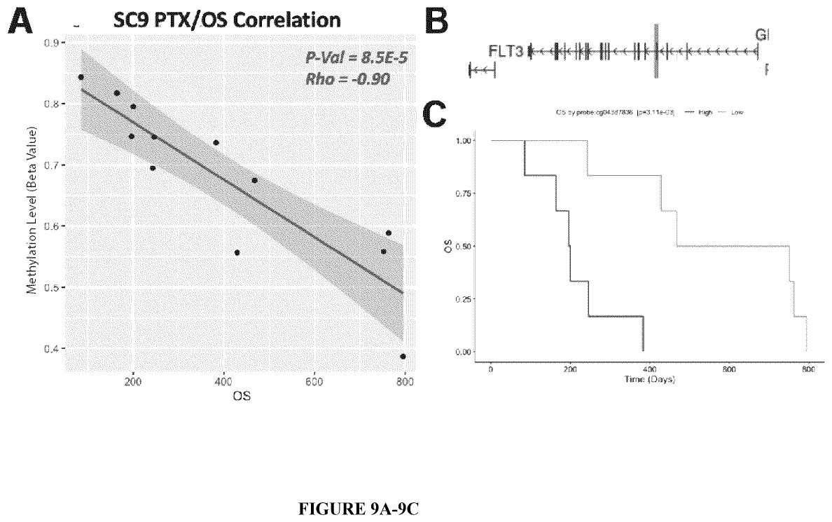

In one embodiment, the cancer-progression metric of interest is subject survival. In one aspect, longer survival is correlated with low methylation of a probe (FIG. 8B, TMEM131 (probe cg16445423); FIG. 9, FLT3 (probe cg04387836)). The trend (positive or negative) in changes in DNA methylation is biomarker dependent.

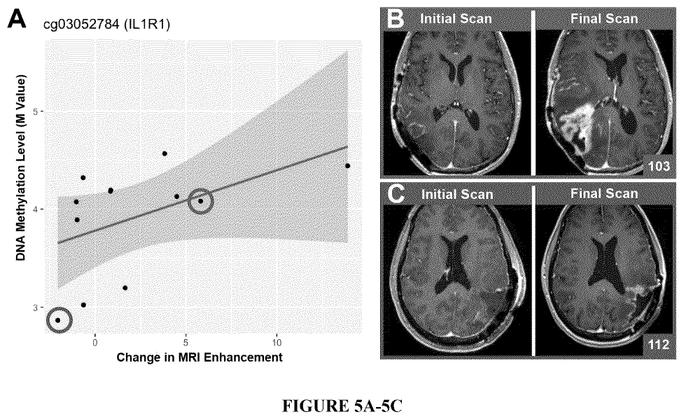

In another embodiment, the cancer-progression metric of interest is change in enhancement between the first and last treatment MRI (ROI enhancement or MRI enhancement). In one aspect, less change in enhancement between the first and last treatment MRI is correlated with lower DNA methylation (FIG. 5). In another aspect, less change in MRI enhancement may be correlated with higher DNA methylation. The trend (positive or negative) in changes in DNA methylation is biomarker gene dependent.

High and low methylation may be user defined, and may be relative to the probe and gene. For example, see the differences in methylation levels between the probes in FIGS. 5A, 8B, and 9A.

In some embodiments, high DNA methylation means a Beta-value of at least 0.75 or greater, at least 0.80 or greater, at least 0.85 or greater, at least 0.90 or greater, or at least 0.95 or greater. In other embodiments, high DNA methylation means different values.

In some embodiments, low DNA methylation means a Beta-value of 0.60 or below, 0.55 or below, 0.5 or below, 0.45 or below, or 0.40 or below. In other embodiments, low DNA methylation means different values.

In some embodiments, high DNA methylation means an M-value of at least 0, at least 1, at least 2, or at least 3. In other embodiments, high DNA methylation means different values.

In some embodiments, low DNA methylation means an M-value of less than 0, less than −1, less than −2, or less than −3. In other embodiments, low DNA methylation means different values.

### Predictive Biomarkers

As disclosed herein, the methods of biomarker detection have identified multiple candidate biomarkers predictive of response to a chemotherapeutic agent.

Exemplary genes that may serve as candidate biomarkers for susceptibility or resistance to cancer therapeutics, such as paclitaxel and/or carboplatin are enumerated in Tables 1-4. These genes were identified with enrichment among resistant cells in genome-wide CRISPR screens in cells exposed to paclitaxel and/or carboplatin.

The genes in Table 5 were identified in the CRISPR screen, and probes within the genes had methylation levels which correlated with patient survival (linear correlation).

For example methylation levels of certain genomic loci within IL1R1, GRIN3B, ZBTB20, EME2, TNRC6C, TMEM131, FLT3, and/or TEX22 may be indicative of patient tumor sensitivity to treatment with a chemotherapeutic agent such as paclitaxel and/or carboplatin.

As described herein, DNA methylation level of one or more biomarkers of the present disclosure may be indicative of sensitivity to treatment with a chemotherapeutic agent such as paclitaxel and/or carboplatin. For example, a lower DNA methylation level of probe cg16445423 (TMEM131) is correlated with longer survival, and a lower DNA methylation level of probe cg03052784 (IL1R1) is correlated with less change in MRI enhancement (FIGS. 5 and 8).

The GRIN3B gene encodes a protein which is a subunit of an N-methyl-D-aspartate (NMDA) receptor. GRIN3B has an mRNA sequence according to NCBI NM_138690.3.

The ZBTB20 gene encodes zinc finger and BTB domain-coding protein 20. ZBTB30 has an mRNA sequence according to NCBI NM_001164342.2.

The EME2 gene encodes the probable crossover junction endonuclease EME2 protein. EME2 has an mRNA sequence according to NCBI NM_001257370.2.

The TNRC6C gene encodes the trinucleotide repeat-containing gene 6C protein. TNRC6C has an mRNA sequence according to NCBI NM_001142640.2.

The TMEM131 gene encodes the transmembrane protein 131. TMEM131 has an mRNA sequence according to NCBI NM_015348.2.

The TEX22 gene encodes the testis-expressed protein 22. TEX22 has an mRNA sequence according to NCBI NM_001195082.2

The FLT3 gene encodes the receptor-type tyrosine-protein kinase FLT3. FLT3 has an mRNA sequence according to NCBI NM_004119.3.

The IL1R1 gene encodes the interleukin-1 receptor type 1. IL1R1 has an mRNA sequence according to NCBI NM_000877.4.

As seen in FIG. 9A, the DNA methylation level (beta value) of probe cg04387836 (gene FLT3) in tissue samples from subjects treated with the chemotherapeutic agent paclitaxel has a negative correlation with overall survival in subjects treated paclitaxel. In other terms, this could be a biomarker of tumor susceptibility to the chemotherapeutic agent paclitaxel, wherein high methylation in a tissue sample indicates worse subject response to paclitaxel and a shorter overall survival.

In some embodiments, a subject's cancer biomarker levels can be determined before, during, and/or after a course of treatment or therapy, or throughout the subject's life.

### Methods of Treatment

According to one embodiment, described herein are a method of treating cancer in a subject. In one aspect the cancer is selected from breast cancer, ovarian cancer, Kaposi's sarcoma, or glioblastoma. In some aspects the cancer is a tumor.

Methods of treatment first include detecting the DNA methylation levels of one or more of the probes for a cancer biomarkers genes in a cancer sample from a subject, where the sample was treated with a chemotherapeutic agent prior to DNA methylation analysis.

The biomarker genes may be selected using the previously described methods. The methods will determine whether DNA methylation indicates cancer susceptibility to the chemotherapeutic agent. The definition of DNA methylation level (i.e. elevated (high) or lower (low)) can be user defined and may be gene dependent. For example, see the differences in methylation levels between the probes in FIGS. 5A, 8B, and 9A.

In some biomarkers, cancer susceptibility to the chemotherapeutic agent may be indicated with an enhanced methylation value. In other biomarkers, cancer susceptibility to the chemotherapeutic agent may be indicated with a lower methylation value. After determining chemotherapeutic agent susceptibility, the at least one chemotherapeutic agent that the cancer is susceptible to is administered to the subject. Chemotherapeutic agents can be administered to the subject as part of a therapeutic composition.

In some embodiments, a biomarker with an elevated methylation level has a Beta-value of at least 0.75 or greater, at least 0.80 or greater, at least 0.85 or greater, at least 0.90 or greater, or at least 0.95 or greater. In other embodiments, elevated DNA methylation means different values.

In some embodiments, a biomarker with a lower methylation level has a Beta-value of 0.60 or below, 0.55 or below, 0.5 or below, 0.45 or below, or 0.40 or below. In other embodiments, lower methylation means different values.

In some embodiments, a biomarker with an elevated methylation level has an M-value of at least 0, at least 1, at least 2, or at least 3. In other embodiments, elevated methylation means different values.

In some embodiments, a biomarker with a lower methylation level has an M value of less than 0, less than −1, less than −2, or less than −3. In other embodiments, lower methylation means different values.

### Compositions

Therapeutic compositions disclosed herein include chemotherapeutic agents such as bleomycin, cyclophosphamide, doxorubicin, epirubicin, etoposide, 5-fluorouracil, methotrexate, oxaliplatin, temozolomide, cisplatin, paclitaxel and/or carboplatin. Such compositions can be formulated and/or administered in dosages and by techniques well known to those skilled in the medical arts taking into consideration such factors as the age, sex, weight, tumor type and stage, condition of the particular patient, and the route of administration.

The compositions may include pharmaceutical solutions comprising carriers, diluents, excipients, preservatives, and surfactants, as known in the art. Further, the compositions may include preservatives (e.g., anti-microbial or anti-bacterial agents such as benzalkonium chloride). The compositions also may include buffering agents (e.g., in order to maintain the pH of the composition between 6.5 and 7.5).

The pharmaceutical compositions may be administered therapeutically. In therapeutic applications, the compositions are administered to a patient in an amount sufficient to elicit a therapeutic effect (e.g., a response which cures or at least partially arrests or slows symptoms and/or complications of disease (i.e., a “therapeutically effective dose”).

In some embodiments, compositions are formulated for systemic delivery, such as oral or parenteral delivery. In some embodiments, minimally invasive microneedles and/or iontophoresis may be used to administer the composition. In some embodiments, compositions are formulated for site-specific administration, such as by injection into a specific tissue or organ, topical administration (e.g., by patch applied to the target tissue or target organ). In some embodiments, the composition is formulated to be delivered through the blood-brain barrier. By way of example, but not by way of limitation, such methods may include ultrasound treatment with or without concomitant administration of microbubbles, convection enhanced drug delivery, biodegradable wafers that release the drug, peptide-drug conjugates, and nanoparticle-drug coupling to enhance drug penetration across the blood-brain barrier.

In some embodiments, the composition formulated for administration comprises between 500 mg/ml and 1000 mg/ml of chemotherapeutic agent. In some embodiments, the composition formulated for administration comprises between 0.1 ng and 500 mg/ml of the chemotherapeutic agent. In some embodiments, the compositions is formulated such that between 0.1 ng and 500 μg/ml of the chemotherapeutic agent is administered to a subject. In some embodiments, the composition is administered at between 500 mg/ml and 1000 mg/ml of chemotherapeutic agent; between 0.1 ng and 500 mg/ml of the inhibitor; or between about 0.1 ng and 500 μg/ml of the chemotherapeutic agent. In some embodiments, at least about 0.1-1.0 μM is administered to a subject. In some embodiments, at least about 0.2-0.7 μM, or 0.3-0.5 μM is administered to a subject.

In some embodiments, the methods include administration of the therapeutic compositions once per day; in some embodiments, the composition may be administered multiple times per day, e.g., at a frequency of one or two times per day, or at a frequency of three or four times per day or more. In some embodiments, the methods include administration of the composition once per week, once per month, or as symptoms dictate.

### Patient Populations

As used herein, the terms “patient,” and “subject” are used interchangeably.

In some embodiments, a subject in need thereof in accordance with the technologies (i.e. methods and compositions) disclosed herein include, but are not limited to, humans and non-human vertebrates. In some embodiments, a subject in need thereof in accordance with the technologies disclosed herein comprise, for example, a mammal. In some embodiments the mammal is a human. In some such embodiments, a mammal includes, for example and without limitation, a household pet (e.g., a dog, a cat, a rabbit, a ferret, a hamster, etc.), a livestock or farm animal (e.g., a cow, a pig, a sheep, a goat, a chicken or another poultry), a horse, a monkey, a laboratory animal (e.g., a mouse, a rat, a rabbit, etc.) and the like. Subjects can also include fish and other aquatic species. In a preferred embodiment, the subject in need thereof in accordance with technologies described herein is a human.

In some aspects, technologies disclosed herein can be practiced in any subject that has (e.g., has been diagnosed with) a brain tumor (e.g., a glioblastoma). A subject having a tumor (e.g., a brain tumor) is a subject that has detectable tumor cells.

In some aspects, technologies of the present disclose can be utilized in a subject that has brain cancer (e.g., glioblastoma) or another cancer. A subject that has cancer is a subject that has detectable cancer cells. In some embodiments, a cancer involves one or more tumors (e.g., brain tumors).

In some aspects, the subject has a cancer selected from breast cancer, ovarian cancer, Kaposi's sarcoma, leukemia, colon cancer, rectal cancer, lung cancer, and glioblastoma.

Tests for diagnosing brain cancers to be treated by technologies described herein are known in the art and can be readily understood and utilized to the ordinary medical practitioner. Such tests include, for example and without limitation, magnetic resonance imaging (MRI), computed tomography (CT) scan, positron emission tomography (PET) scan, diagnostic angiogram, myelogram, etc. Generally, medical practitioners also take a full medical history and conducts a complete physical examination in addition to the tests listed above.

In some aspects, technologies of the present disclosure can be utilized in a subject that has a glioblastoma (e.g., glioblastoma multiforme GBM). Methylation of the promoter region of the DNA repair enzyme gene, O6-methylguanine-DNA methyltransferase (MGMT), is a prognostic factor for glioblastoma. MGMT removes alkyl groups from guanine in the DNA. Without wishing to be bound by any one theory, it is understood that MGMT removal of alkyl groups from guanine in the DNA counteracts the therapeutic efficacy of alkylating chemotherapeutics, such as temozolomide (TMZ). Epigenetic silencing (e.g., methylation) of the promoter region of MGMT might lead to transcriptional repression and a decreased MGMT protein expression and is associated with an improved response to TMZ chemotherapy and longer overall survival of GBM patients. In some embodiments, a subject that has glioblastoma comprises a methylated MGMT promoter. In some such embodiments, a subject that has glioblastoma comprises an unmethylated MGTM promoter. MGMT methylation can be assessed by a plurality of methods known in the art, including, for example, by methylation-specific PCR (MSP), high-resolution melting PCR (HRM), or pyrosequencing. A cut-off value of 8-10% can distinguish unmethylated from methylated MGMT promoters. See, e.g., Feldheim J. et al., Cancers (Basel). 2019 Nov. 21; 11(12):1837.

In some aspects, the subject in need thereof in accordance with technologies of the present disclosure has undergone one or more other cancer therapies (e.g., chemotherapy, radiotherapy, immunotherapy). In some embodiments, the subject in need thereof has previously undergone or more other cancer therapies and the subject's cancer has relapsed. In some embodiments, the subject in need thereof has previously undergone one or more other cancer therapies and the subject has developed resistance to the one or more other cancer therapies. In some embodiments, the subject in need thereof is in remission (e.g., partial remission, complete remission). In some embodiments, the subject in need thereof is refractory to one or more other cancer therapies.

In some embodiments, the subject in need thereof is an adult. In some embodiments, the subject in need thereof is a human subject over 18 years of age. In some embodiments, the subject in need thereof is a human subject over 21 years of age. In some embodiments, the subject in need thereof is a human subject over 30 years of age. In some embodiments, the subject in need thereof is a human subject over 65 years of age. In some embodiments, the subject in need thereof is a human subject under 18 years of age. In some embodiments, the subject in need thereof is a human subject under 65 years of age (or between 18 and 65 years of age, between 21 and 65 years of age, or between 30 and 65 years of age). In some embodiments, the subject in need thereof is a pediatric subject (e.g., a human subject under the age of 12).

### EXAMPLES

The following examples are illustrative and should not be interpreted to limit the scope of the claimed subject matter.

### Summary

Paclitaxel and carboplatin are chemotherapies used for the treatment of glioblastoma and other forms of cancer. Susceptibility to these drugs is variable among patients, and pre-treatment prediction of tumor sensitivity is essential to optimize patient selection and therapeutic efficacy. Whereas for paclitaxel, Applicant previously identified genes that confer susceptibility through CRISPR screens (see Dmello 2022), development of a predictive biomarker that is testable on the tumor tissue is limited by the reproducibility of staining, CLIA certification of a test, and the contribution of several genes to the susceptibility phenotype, which are difficult to assess simultaneously. Applicant performed genome-wide knock-out screens in glioma cells to identify genes that confer sensitivity to paclitaxel, carboplatin or combined treatment in a patient-derived glioma xenograft line. Targets exhibiting the highest growth advantage after knock-out were further investigated in clinical trial patients receiving these therapies after opening of the blood-brain barrier (Sonocloud 9 clinical trial; NCT04528680). Applicant investigated DNA methylation signals in genomic loci that might regulate these genes, and correlated patient survival with levels of each modality. Consequently, Applicant's approach identified functional genes associated with paclitaxel/carboplatin response in vitro, and related gene regulatory features that correlate with outcomes such as local disease control and overall survival in prospective clinical trial patients treated with these drugs.

Through use of a DNA methylation platform that is already CLIA-certified, Applicant can interrogate these biomarkers using the clinical standard-of-care pipeline used by pathologists to analyze brain tumors.

In short, Applicant is able to analyze DNA methylation values in cancer samples exposed to chemotherapeutic agents and compare to known methylation patterns of biomarker genes in cancer samples exposed to the chemotherapeutic agent to determine whether a cancer will be susceptible to treatment with the chemotherapeutic agent.

### Methods and Results

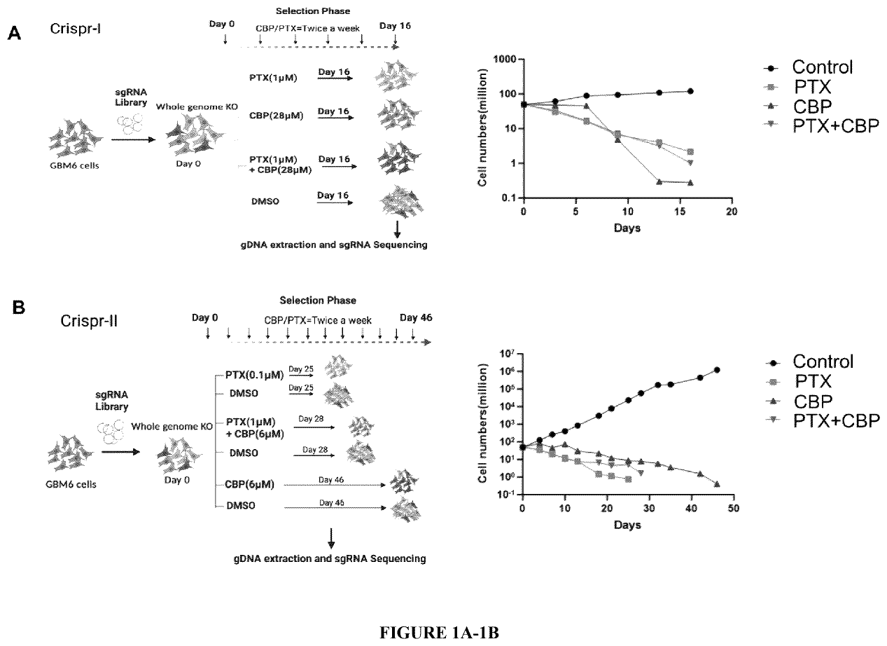

Genome-wide clustered regularly interspaced short palindromic repeats (CRISPR) knock-out screens were performed to identify candidate genes that confer sensitivity to paclitaxel, carboplatin, or combination therapy (FIG. 1). Described herein are results from 2 CRISPR screens: an earlier paclitaxel only screen (Dmello 2022, PMID35552677) as well as a new screen with paclitaxel, carboplatin, or combination.

Experiments in the earlier paclitaxel screen were performed in-vitro using the glioma cell line “H4,” grown in in Dulbecco's Minimum Essential Media (DMEM) Experiments in the containing 10% FBS. In that screen, DNA was extracted from treated cells 21 days after treatment. DNA was extracted from the surviving cell using the Zymo Research Quick-DNA midiprep plus kit, and amplified with the unique barcode primer.

Experiments in the other screen were performed in vitro using the glioblastoma cell line GBM6. Each clonal cell population (harboring a single targeted gene) then underwent twice weekly treatment with paclitaxel (PTX), carboplatin (CBP), combination, or control (DMSO), to provide selection pressure on the cells. CRISPR drug screens were performed at (FIG. 1A) high and (FIG. 1B) low drug concentrations to compare the effects. The high dose of paclitaxel was 1 μM and the high dose of carboplatin was 28 μM. The low dose of paclitaxel was 0.1 μM and the low dose of carboplatin was 6 μM. After 16 days, cells in the high concentration experiment were harvested and underwent sequencing to calculate which genes were enriched after selection (i.e. determine sensitivity-conferring genes for each drug). Depending on the schematic in FIG. 1B, cells in the low concentration experiment were harvested at either 25, 28, or 46 days. The harvested cells underwent sequencing to calculate which genes were enriched after selection. The low concentration experiments were harvested at different times because the chemotherapeutic treatment targeted the cell populations at different rates (as seen in the cell number graphs in FIG. 1B).

The Brunello sgRNA library was utilized to target genes, which includes 70,000 sgRNAs with 3-4 sgRNA per gene, plus 10,000 gRNA non-targeting controls. Other experiments were performed in vitro using the glioblastoma cell line GBM6.

Library preparation, virus production and multiplicity of infection (MOI) determination was done as described in PMID28333914. Fifty million cells were treated for each experimental condition (paclitaxel, carboplatin, combination, or PBS). Both CRISPR screens were pooled screens.

After standard library preparation, samples underwent sequencing on the Illumina platform aiming for a coverage of >1000 reads per sgRNA in the library.

In the earlier paclitaxel screen (Dmello 2022), data analysis was performed with the bioinformatics tool CRISPRAnalyzeR. DEseq and sgRSEA algorithms were used to identify enriched genes (FIG. 2). Guides with raw read counts <40 were excluded from the analysis. Analysis of paclitaxel in H4 cells identified 51 genes with enrichment among resistant cells (Table 1), suggesting a possible role for these genes in conferring paclitaxel sensitivity.

TABLE 1
Genes with enrichment among resistant cells in the earlier paclitaxel
screen. Genes were identified using DESeq and sgRSEA to identify
enriched genes from the pooled CRISPR-mutant paclitaxel screen
(identified in Dmello et al 2022, doi: 10.1158/1078-0432.CCR-
21-2563). Genes with enrichment are also viewed in FIG. 2.

| sgRSEA R enriched | Deseq enriched |
|---|---|
| RSPO3 | GOLGA8J |
| CEP63 | KTI12 |
| USP28 | PSPC1 |
| NEK5 | SEMA5A |
| FLT3 | MUC13 |
| LMAN2L | TMEM131 |
| TP53 | ZNF813 |
| MYD88 | NPS |
| EAF2 | SLC35C1 |
| IL1R1 | IRAK4 |
| TRNP1 | SMCR8 |
| ERG | EPC2 |
| TDRD1 | GSDMC |
| TEX22 | GRIN3B |
| SSR3 | NPPC |
| CCNE1 | RELA |
| IRAK1 | UCHL5 |
| EME2 | SDF2 |
| PPP3CA | IRAK1 |
| HMCN2 | TARBP2 |
| TNFRSF10B | PQLC3 |
| MRAS | FGFBP3 |
| IL1RAP | TEX22 |
| TNRC6C | ECEL1 |
| ASCL2 | MBNL1 |
| SMAGP | |
| UNC119 | |
| ZBTB20 | |

In the GBM6 cell line screen (paclitaxel, carboplatin, or both), analysis identified 88 genes that were enriched after exposure to carboplatin; 69 genes that were enriched after exposure to paclitaxel; and 72 genes that were enriched after exposure to both carboplatin and paclitaxel (high concentration, as seen in FIG. 1) (FIG. 3D). Gene enrichment was presented as the Beta score (not to be confused with the ‘beta’ methylation level described above), which is the relative enrichment of gene sgRNAs in the chemotherapeutic agent treatments as compared to the control). A gene with a high Beta score (approximately 2 or greater) was considered enriched (FIGS. 3A-3C). Enriched genes in the GBM6 cells exposed to paclitaxel, carboplatin, or a combination are in Tables 2-4.

TABLE 2
Susceptible genes in GBM6 cells exposed to paclitaxel
at a high concentration. Three to four sgRNAs were
used for each gene, and indicated in the table is which
sgRNA for a gene was enriched. 69 genes enriched.

| | | | Paclitaxel_false |
|---|---|---|---|
| | | Paclitaxel_beta | discovery rate |
| Gene | sgRNA | value | of the gene |
| UGP2 | 3 | 2.846752 | 0 |
| C1orf109 | 3 | 2.746872 | 0 |
| ARHGEF38 | 2 | 2.659702 | 0.010989 |
| BFSP1 | 2 | 2.62215 | 0.018692 |
| HEMGN | 2 | 2.620504 | 0.010989 |
| MIS18A | 2 | 2.512567 | 0.018692 |
| VWA5A | 3 | 2.508285 | 0 |
| SAAL1 | 2 | 2.484745 | 0.018692 |
| BMS1 | 2 | 2.479748 | 0.018692 |
| FAM98A | 2 | 2.468491 | 0.018692 |
| DMRTA2 | 2 | 2.462597 | 0.018692 |
| RHPN1 | 3 | 2.456323 | 0 |
| TICRR | 2 | 2.451593 | 0.025424 |
| KCND1 | 2 | 2.44875 | 0.018692 |
| PTGES | 3 | 2.423852 | 0 |
| SFRP5 | 2 | 2.41878 | 0.025424 |
| EFCAB11 | 4 | 2.397905 | 0 |
| ANAPC15 | 2 | 2.395387 | 0.032051 |
| C16orf58 | 3 | 2.388995 | 0 |
| IL37 | 3 | 2.369412 | 0 |
| UPK1B | 4 | 2.367048 | 0 |
| KIAA0391 | 2 | 2.349816 | 0.025424 |
| FPGT | 4 | 2.329036 | 0 |
| CALML6 | 3 | 2.294927 | 0 |
| CDC27 | 2 | 2.294361 | 0.036364 |
| HSPA14 | 3 | 2.289557 | 0 |
| SSBP1 | 3 | 2.276759 | 0 |
| DHX16 | 3 | 2.273981 | 0 |
| ATP8B4 | 3 | 2.267182 | 0 |
| KIF3A | 3 | 2.263648 | 0 |
| ZNF185 | 3 | 2.263579 | 0 |
| C14orf2 | 4 | 2.246982 | 0 |
| SRSF1 | 2 | 2.241737 | 0.038732 |
| FLI1 | 2 | 2.24106 | 0.040134 |
| B4GALT7 | 3 | 2.210013 | 0 |
| ATP6V1E1 | 3 | 2.20831 | 0 |
| CTU2 | 3 | 2.198218 | 0 |
| KDSR | 3 | 2.172225 | 0 |
| SPATA31A3 | 3 | 2.168528 | 0 |
| GPR158 | 2 | 2.168445 | 0.049133 |
| GLYR1 | 4 | 2.164112 | 0.025424 |
| OR4F4 | 2 | 2.161351 | 0.050398 |
| KNOP1 | 4 | 2.160306 | 0.018692 |
| CETN2 | 2 | 2.143861 | 0.042763 |
| PLAGL1 | 2 | 2.142826 | 0.047904 |
| SCG2 | 3 | 2.125094 | 0 |
| CPNE5 | 3 | 2.120108 | 0 |
| MANEAL | 3 | 2.102125 | 0 |
| TROAP | 3 | 2.10008 | 0.010989 |
| TTC39A | 4 | 2.100072 | 0.025424 |
| PSMB1 | 3 | 2.087841 | 0 |
| GAN | 3 | 2.07733 | 0 |
| SORBS1 | 4 | 2.066245 | 0.029851 |
| RANBP2 | 2 | 2.064817 | 0.050398 |
| CACNB2 | 3 | 2.064368 | 0.010989 |
| CKS2 | 3 | 2.058656 | 0.010989 |
| RALY | 3 | 2.057626 | 0.010989 |
| UIMC1 | 3 | 2.052654 | 0.010989 |
| MEX3A | 3 | 2.040517 | 0.010989 |
| MATK | 4 | 2.040204 | 0.029851 |
| RBFA | 3 | 2.035791 | 0.010989 |
| CTDSPL2 | 3 | 2.033 | 0.010989 |
| VPS37D | 3 | 2.032278 | 0.010989 |
| IFNG | 4 | 2.027424 | 0.033898 |
| OR4F17 | 3 | 2.015997 | 0.010989 |
| MRPL35 | 3 | 2.009868 | 0.010989 |
| CYB561D2 | 3 | 2.009711 | 0.010989 |
| NBEAL1 | 4 | 2.007339 | 0.033898 |
| COMMD1 | 4 | 2.00082 | 0.025424 |

TABLE 3
Susceptible genes in GBM6 cells exposed to carboplatin
at a high concentration. Three to four sgRNAs were
used for each gene, and indicated in the table is which
sgRNA for a gene was enriched. 88 genes enriched.

| | | Carboplatin_beta | Carboplatin_false |
|---|---|---|---|
| Gene | sgRNA | value | discovery rate |
| POLR2I | 2 | 2.74667 | 0 |
| SNAP29 | 2 | 2.737866 | 0 |
| HIST1H2AI | 2 | 2.712235 | 0 |
| ARMCX1 | 2 | 2.676202 | 0 |
| ZSWIM3 | 2 | 2.631248 | 0 |
| LMNA | 2 | 2.591395 | 0 |
| RAPGEF6 | 2 | 2.570867 | 0.014925 |
| SNX18 | 3 | 2.520684 | 0 |
| RHOB | 2 | 2.44657 | 0.033333 |
| UQCR10 | 4 | 2.441567 | 0.014925 |
| GOLGA4 | 3 | 2.435534 | 0 |
| SORBS2 | 4 | 2.428328 | 0.014925 |
| AP2A2 | 4 | 2.408583 | 0.014925 |
| NBPF3 | 3 | 2.395647 | 0 |
| XKRY2 | 2 | 2.377058 | 0.042904 |
| HLX | 2 | 2.344389 | 0.032558 |
| PPP4C | 2 | 2.334654 | 0.042904 |
| TMEM254 | 3 | 2.332227 | 0 |
| ATXN2 | 4 | 2.326182 | 0.032258 |
| BCLAF1 | 2 | 2.320276 | 0.035714 |
| NOP14 | 3 | 2.303174 | 0 |
| POLN | 3 | 2.298726 | 0 |
| TDP1 | 3 | 2.295357 | 0 |
| MRPL39 | 3 | 2.288407 | 0.014925 |
| CXorf49 | 2 | 2.287516 | 0.037736 |
| MEPCE | 2 | 2.285811 | 0.033333 |
| HIATL1 | 2 | 2.282175 | 0.037736 |
| PTH | 4 | 2.279482 | 0.014925 |
| RASSF8 | 4 | 2.276224 | 0.032258 |
| FTSJ2 | 2 | 2.273006 | 0.047904 |
| LYAR | 4 | 2.272361 | 0.032258 |
| RPL8 | 3 | 2.265096 | 0.014925 |
| MATK | 4 | 2.259106 | 0.014925 |
| HIST1H1B | 3 | 2.258828 | 0.014925 |
| OR4F5 | 2 | 2.256973 | 0.035714 |
| CCDC136 | 3 | 2.239029 | 0 |
| CST6 | 3 | 2.233157 | 0.014925 |
| GCKR | 2 | 2.232227 | 0.035714 |
| SLC38A11 | 4 | 2.228947 | 0.032258 |
| FBXO48 | 2 | 2.227023 | 0.04717 |
| MYL10 | 2 | 2.222248 | 0.037736 |
| PIP5K1A | 3 | 2.220422 | 0.014925 |
| TSPAN13 | 4 | 2.214487 | 0.032258 |
| OXA1L | 3 | 2.214269 | 0 |
| RBFA | 3 | 2.209177 | 0.014925 |
| CRTAM | 3 | 2.203994 | 0 |
| SEC23A | 4 | 2.185581 | 0.032258 |
| MAD1L1 | 4 | 2.18531 | 0.032258 |
| UXT | 2 | 2.182573 | 0.042904 |
| GFI1B | 3 | 2.181772 | 0.014925 |
| TTI2 | 2 | 2.174897 | 0.059289 |
| C21orf58 | 3 | 2.171661 | 0.014925 |
| AGAP4 | 2 | 2.164251 | 0.04717 |
| WDR63 | 3 | 2.162149 | 0.014925 |
| NSD1 | 2 | 2.155757 | 0.042904 |
| NR2C2AP | 3 | 2.147639 | 0.014925 |
| CYFIP1 | 3 | 2.144852 | 0.014925 |
| ATP5O | 3 | 2.120334 | 0.014925 |
| TCP1 | 2 | 2.11559 | 0.048426 |
| RD3L | 4 | 2.113214 | 0.032258 |
| SCG2 | 3 | 2.112166 | 0.014925 |
| OR1B1 | 4 | 2.105201 | 0.032558 |
| SLX4 | 3 | 2.101022 | 0.014925 |
| SSBP1 | 3 | 2.097276 | 0.014925 |
| DDB2 | 4 | 2.095241 | 0.032258 |
| VCP | 2 | 2.091599 | 0.048426 |
| SLC46A2 | 2 | 2.086772 | 0.048387 |
| WDR75 | 4 | 2.085588 | 0.032558 |
| TASP1 | 4 | 2.085129 | 0.032558 |
| GPR158 | 2 | 2.083633 | 0.048426 |
| PSMC6 | 3 | 2.074891 | 0.014925 |
| ERO1L | 3 | 2.072793 | 0.014925 |
| NSFL1C | 3 | 2.062733 | 0.026316 |
| STAT2 | 4 | 2.055189 | 0.032258 |
| TCTN1 | 3 | 2.042853 | 0.014925 |
| ROBO2 | 3 | 2.034303 | 0.014925 |
| POLR3C | 2 | 2.03244 | 0.05298 |
| ZNF185 | 3 | 2.031312 | 0.014925 |
| CLDN25 | 3 | 2.031268 | 0.014925 |
| PRY | 2 | 2.028781 | 0.05298 |
| MOGS | 4 | 2.02611 | 0.032258 |
| ZNF445 | 3 | 2.014723 | 0.014925 |
| OR13F1 | 3 | 2.012083 | 0.032558 |
| NBPF7 | 3 | 2.011198 | 0.032258 |
| UBE4B | 3 | 2.008344 | 0.014925 |
| CCDC12 | 3 | 2.00394 | 0.026316 |
| SCN5A | 4 | 2.000133 | 0.032258 |
| NDUFA4 | 4 | 2.000096 | 0.032558 |

TABLE 4
Susceptible genes in GBM6 cells exposed to both carboplatin
and paclitaxel at high concentrations. Three to four
sgRNAs were used for each gene, and indicated in the
which sgRNA for a gene was enriched. 72 genes enriched.

| | | Combination_beta | Combination_false |
|---|---|---|---|
| Gene | sgRNA | value | discovery rate |
| NSMCE2 | 2 | 3.394291549 | 0 |
| RPS14 | 3 | 3.274283512 | 0 |
| PSMD7 | 2 | 3.197951157 | 0 |
| FBXO48 | 2 | 3.188628486 | 0 |
| CLDN25 | 3 | 3.066436844 | 0 |
| ATP5C1 | 3 | 3.016679992 | 0 |
| MRGBP | 3 | 2.988590036 | 0 |
| ZNF235 | 3 | 2.848581848 | 0 |
| CTU2 | 3 | 2.794118976 | 0 |
| UTF1 | 2 | 2.724709193 | 0.028986 |
| OR4F5 | 2 | 2.690187332 | 0.028986 |
| XKRY2 | 2 | 2.670922221 | 0.04 |
| GPR107 | 2 | 2.608360623 | 0.04 |
| DR1 | 3 | 2.598567809 | 0 |
| RHOB | 2 | 2.547736175 | 0.045113 |
| DNMT1 | 3 | 2.543027735 | 0 |
| SMC4 | 2 | 2.535053012 | 0.04 |
| HSPB1 | 3 | 2.509396649 | 0 |
| ILK | 3 | 2.485156741 | 0 |
| SNAI1 | 3 | 2.479673563 | 0 |
| KLHL1 | 2 | 2.463862779 | 0.045113 |
| UBE2C | 3 | 2.458835694 | 0.020408 |
| C7orf57 | 4 | 2.456261519 | 0 |
| OR4F4 | 2 | 2.443048487 | 0.040541 |
| RPL8 | 3 | 2.44047062 | 0.020408 |
| RNF212 | 3 | 2.436433372 | 0 |
| NOLC1 | 2 | 2.399592123 | 0.045113 |
| CASC1 | 3 | 2.392501153 | 0.020408 |
| AGAP4 | 2 | 2.380886254 | 0.045113 |
| CCDC136 | 3 | 2.378310201 | 0 |
| CDKL3 | 3 | 2.371459429 | 0 |
| KRTCAP3 | 4 | 2.368957052 | 0.020408 |
| RANBP2 | 2 | 2.355915311 | 0.040541 |
| TCP11L1 | 4 | 2.352136448 | 0 |
| CST6 | 3 | 2.339668721 | 0.020408 |
| CYP2C8 | 3 | 2.314707329 | 0.020408 |
| OR4F17 | 3 | 2.314273404 | 0 |
| RBFA | 3 | 2.314254262 | 0.020408 |
| HTR4 | 4 | 2.312207662 | 0.028986 |
| OBSCN | 2 | 2.311163103 | 0.045113 |
| UTP3 | 3 | 2.311067546 | 0.028986 |
| ZMYM1 | 3 | 2.304338002 | 0.020408 |
| TSPAN13 | 4 | 2.303340679 | 0.028986 |
| ATXN2 | 4 | 2.296471422 | 0.028986 |
| CYP4X1 | 4 | 2.280370359 | 0.028986 |
| ERCC3 | 3 | 2.268743507 | 0 |
| VPS28 | 3 | 2.249272197 | 0.035294 |
| TBL1X | 4 | 2.22467918 | 0.028986 |
| RHOQ | 2 | 2.212587767 | 0.047337 |
| PCDHB11 | 4 | 2.206645263 | 0.028986 |
| FEZF1 | 3 | 2.200951875 | 0.035294 |
| CABP1 | 2 | 2.199921017 | 0.056433 |
| MYL10 | 2 | 2.188532791 | 0.047337 |
| GPR61 | 4 | 2.188454725 | 0.028986 |
| EPB41L5 | 4 | 2.180201258 | 0.028986 |
| NDUFA4 | 4 | 2.128192816 | 0.028986 |
| TCTN1 | 3 | 2.116475124 | 0.036697 |
| AKTIP | 4 | 2.088586107 | 0.028986 |
| KIAA1210 | 4 | 2.085584387 | 0.028986 |
| TASP1 | 4 | 2.06618171 | 0.035294 |
| METTL10 | 4 | 2.060496605 | 0.028986 |
| RSC1A1 | 3 | 2.058642601 | 0.036697 |
| CD96 | 3 | 2.055397492 | 0.035294 |
| DEFB127 | 4 | 2.053982153 | 0.036697 |
| ACP2 | 3 | 2.028042122 | 0.035294 |
| ATAT1 | 3 | 2.026607703 | 0.036697 |
| CCDC12 | 3 | 2.023509649 | 0.036697 |
| WDR5 | 3 | 2.021685324 | 0.035294 |
| SLC46A2 | 2 | 2.021473156 | 0.059794 |
| SLC9A3 | 4 | 2.020649771 | 0.040541 |
| TMEM254 | 3 | 2.020306301 | 0.04 |
| GLYR1 | 4 | 2.016791297 | 0.035294 |

To determine which genes identified in the CRISPR are contributors to susceptibility and may serve as predictive biomarkers in patients, Applicant used DNA methylation data from patients enrolled in the Sonocloud 9 clinical trial (NCT04528680). For example, patients in the completed phase I trial received treatment with paclitaxel (n=17), enabling comparison of overall survival and local control with respect to their epigenetic and transcriptional regulation in the same patients. DNA methylation data was obtained using the Illumina EPIC 850K array. Standard processing pipelines were used. For each treatment type, candidate genes identified from the respective CRISPR screen were narrowed to increase power for validation. For the DNA methylation analysis, this included any genes in which (i) less than 3 probes are available for consideration in the gene promotor, (v) the average promotor probe failure is greater than 25%, or (vi) average intensity differences between the upper and lower quartile of samples is less than 10%.

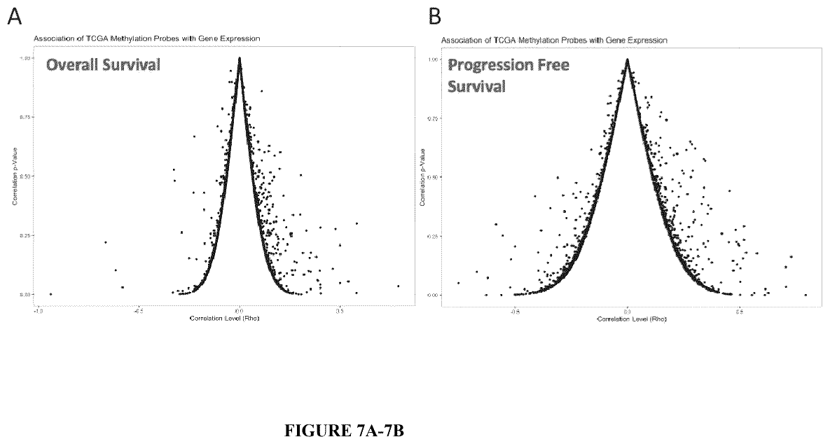

Candidate DNA methylation probes that might regulate the identified genes underwent correlation of DNA methylation level with overall survival in the Sonocloud clinical trial for patients that were treated with the maximal tolerated dose of paclitaxel, using ultrasound-based blood-brain barrier opening to enhance drug penetration. Methylation correlation was performed after pre-processing using the SESAME pipeline. Methylation probes are correlated with gene expression (FIG. 6). Applicant also found that methylation probes for candidate biomarker genes did not correlate with overall survival or progression free survival (FIG. 7, correlating the 51 genes in the first paclitaxel screen with overall survival and progression free survival in glioblastoma patients). That is, gene methylation level of the candidate biomarkers was not prognostic of survival in the absence of chemotherapeutic treatment.

Applicants identified 25 probes that were most correlated with gene expression and fell within 15 kb of a sensitivity gene candidate identified from the paclitaxel screen (first paclitaxel screen, Table 1 and FIG. 2). The cut-off for these probes is a false discovery rate (FDR) q value of <0.05. The list of probes is below, in Table 5. Some candidate genes have multiple probes correlated with gene expression.

Some candidate genes were not included in this list of 25 probes, and applicant further identified probes corresponding with those genes (see e.g. FIG. 5, which shows correlation data for a probe corresponding to IL1R1 and FIG. 9, which shows correlation data for a probe corresponding to FLT3).

TABLE 5
Probes (and corresponding genes) which correlate
with gene expression. FDR q <0.05. Candidate genes

| | | FDR Corrected | |
|---|---|---|---|
| Gene | ProbeID | P-Value | Rho |
| IL1RAP | cg08319289 | 8.27154E−11 | −0.5418226 |
| TNFRSF10B | cg11702403 | 2.81209E−10 | −0.5321921 |
| CEP63 | cg04001268 | 1.18904E−08 | −0.5019898 |
| MYD88 | cg02829783 | 3.02668E−07 | −0.475654 |
| MYD88 | cg24710904 | 3.25623E−07 | −0.471109 |
| TNFRSF10B | cg13580286 | 5.0491E−07 | −0.4667753 |
| TNRC6C | cg00423030 | 1.18555E−06 | −0.458164 |
| IL1RAP | cg02007927 | 5.33258E−06 | −0.4435948 |
| ZBTB20 | cg00097432 | 1.53132E−05 | −0.4308164 |
| MYD88 | cg15427004 | 2.09044E−05 | −0.4261506 |
| MYD88 | cg01353464 | 0.000168351 | −0.4039043 |
| TEX22 | cg08726045 | 0.001399044 | 0.37566907 |
| ZNF813 | cg12142445 | 0.001427553 | −0.3786087 |
| MYD88 | cg17034030 | 0.002675124 | −0.3668557 |
| ZBTB20 | cg03572284 | 0.004037742 | −0.3601009 |
| MBNL1 | cg03333737 | 0.005448908 | −0.3578884 |
| IRAK1 | cg22194593 | 0.006768864 | −0.353759 |
| ZBTB20 | cg14338114 | 0.011484539 | −0.3460313 |
| TMEM131 | cg16445423 | 0.016488464 | −0.344583 |
| UCHL5 | cg24061886 | 0.01823911 | −0.3400706 |
| TDRD1 | cg19002907 | 0.021135703 | −0.3368526 |
| ASCL2 | cg00318682 | 0.041998861 | 0.32900341 |
| SEMA5A | cg12676289 | 0.044234884 | 0.3253324 |

Additional probes will be identified during analysis of the other chemotherapeutic agent screen (carboplatin, paclitaxel, or both, Tables 2-4).

Differentially methylated regions were calculated among candidate genes based on the stratification of samples based on overall survival, progression free survival, or change in enhancement between the first and last treatment MRIs (“ROIEnhanceDelta”) (FIGS. 5, 8-9). Methylation levels of probes within candidate genes were correlated with each metric using the Pearson correlation coefficient.

The best biomarker candidates have linear correlations between the methylation levels and metric. However, the correlation may be positive or negative, depending on the biomarker.

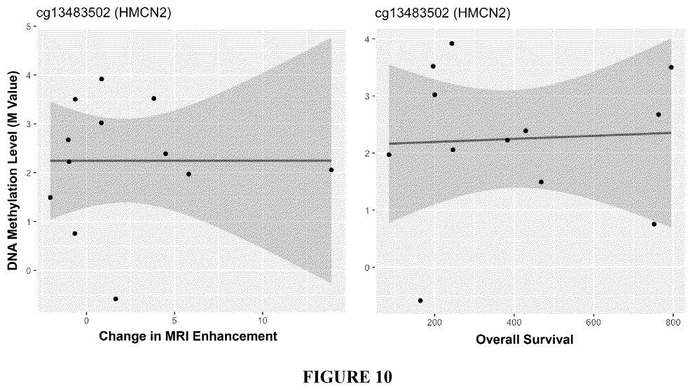

Not all probes associated with a candidate susceptibility gene correlated with a cancer progression metric of interest. For example, FIG. 10 shows two probes associated with gene HMCN2, which have no correlation despite HMCN2 being identified as a candidate gene in the first paclitaxel screen (Table 1 and FIG. 2).

Select results for correlation between ROI enhancement and DNA methylation values for paclitaxel treatment are shown in Table 6.

Methylation values (M value) from a candidate probe falling within gene IL1R1, are shown in FIG. 5A and compared to level of enhancement change (x-axis). A linear relationship is observed between DNA methylation values and ROI enhancement.

Further, as shown in FIG. 8, methylation of probe cg16445423 (gene TMEM131) (M value) was correlated with both gene expression and overall survival of cancer patients treated with paclitaxel.

In FIG. 9A, Applicant demonstrated correlation between the probe cg04387836 (gene FLT3) with overall survival after treatment with paclitaxel, while FIG. 9C shows the Kaplan-Meier curve for samples stratified based on the median methylation value for this probe. Patients with less methylation of the probe had overall increased survival time as compared to patients with more methylation of the probe.

TABLE 6
Correlation of DNA Methylation Probes with Change in
ROI Enhancement, which is enhancement between the first
and last treatment MRIs in a patient in patients treated
with paclitaxel. Some genes, such as GRIN3B, have multiple
probes which correlate with change in ROI enhancement.
These genes were all identified as susceptible in the
first paclitaxel screen (Table 1).

| Gene | Probe_ID | Effect Size | p-value | FDR q-value |
|---|---|---|---|---|
| GRIN3B | cg15471372 | −0.0163 | 0.0001 | 0.07654855 |
| ZBTB20 | cg14941895 | −0.0141 | 0.0008 | 0.42224191 |
| EME2 | cg01676844 | −0.0162 | 0.0164 | 1 |
| TNRC6C | cg02283662 | −0.0151 | 0.0038 | 1 |
| GRIN3B | cg02922913 | −0.0130 | 0.0123 | 1 |
| TEX22 | cg02970735 | −0.0154 | 0.0308 | 1 |
| GRIN3B | cg03328615 | −0.0060 | 0.0274 | 1 |

TABLE 7
Genes conferring sensitivity to Carboplatin (28 uM) in
GBM6 cells in the second round of screening. Genes were
identified using the CB-2 algorithm and filtered to included
genes with log2 fold change of greater than 1.25 and adjusted
P value (FDR_PB) of less than 0.05.

| gene | logFC | fdr_pb |
|---|---|---|
| OR6B1 | 1.68874798 | 0.04431769 |
| CPB2 | 1.68407351 | 0.04622997 |
| LEUTX | 1.68330882 | 0.04758678 |
| CNFN | 1.62068853 | 0.04681734 |
| OR4D11 | 1.55408707 | 0.02773866 |
| ZNF814 | 1.55268276 | 0.04159558 |
| RARRES3 | 1.54077166 | 0.04115334 |
| PGPEP1 | 1.540541 | 0.04369352 |
| IFNL1 | 1.53591578 | 0.03187885 |
| LYZL4 | 1.52822381 | 0.04261197 |
| PCDHGB6 | 1.5148862 | 0.04470394 |
| S100B | 1.50124569 | 0.03325218 |
| EMD | 1.49952082 | 0.02773866 |
| TMED7- | 1.49250277 | 0.03187885 |
| TICAM2 | | |
| DNAJC18 | 1.4873623 | 0.03857089 |
| LOC100129924 | 1.4821928 | 0.04016287 |
| ZNF780B | 1.47180091 | 0.02773866 |
| CUBN | 1.46104561 | 0.04101792 |
| DAOA | 1.45876154 | 0.03959362 |
| ACRBP | 1.45862383 | 0.03541679 |
| APCS | 1.44811397 | 0.04996638 |
| NUDT22 | 1.43795824 | 0.04758678 |
| DNAH1 | 1.43791564 | 0.04868739 |
| MIER1 | 1.43422115 | 0.03886741 |
| CD207 | 1.42679128 | 0.03857089 |
| ATP4A | 1.42381909 | 0.02773866 |
| BOD1L1 | 1.41022013 | 0.04068179 |
| TLL1 | 1.40593667 | 0.04910582 |
| NKX2-1 | 1.4057728 | 0.04068179 |
| PPM1F | 1.40082046 | 0.04233917 |
| FBXL19 | 1.39755642 | 0.04910582 |
| PDSS1 | 1.39105591 | 0.03857089 |
| CFAP47 | 1.38072896 | 0.02949601 |
| SLC25A42 | 1.3751079 | 0.04115334 |
| POLE4 | 1.36866724 | 0.03540154 |
| ALX3 | 1.3628206 | 0.04352197 |
| HRASLS2 | 1.35989782 | 0.04910582 |
| EHMT1 | 1.35749341 | 0.03595259 |
| PIK3R3 | 1.35590103 | 0.04969771 |
| ACPT | 1.35192221 | 0.04954506 |
| SRRD | 1.34996975 | 0.04910582 |
| CXCL17 | 1.34915915 | 0.04697472 |
| ZFAND4 | 1.34432955 | 0.04370908 |
| SLC28A3 | 1.34325708 | 0.04613068 |
| WDR83OS | 1.34257986 | 0.0469631 |
| LOC643355 | 1.33731717 | 0.04910582 |
| PPM1K | 1.33605189 | 0.03168948 |
| QRFPR | 1.32693368 | 0.04261197 |
| SCNN1A | 1.32656036 | 0.04101792 |
| ARHGAP24 | 1.32602999 | 0.04158546 |
| TNFSF10 | 1.32478722 | 0.0410537 |
| IRGQ | 1.32445448 | 0.02773866 |
| ZBTB7C | 1.31702249 | 0.0428167 |
| PTGDR2 | 1.3169813 | 0.033227 |
| HSD11B2 | 1.31665652 | 0.04369352 |
| CRCT1 | 1.31492868 | 0.03777454 |
| CD200 | 1.30514738 | 0.02499178 |
| ZNF530 | 1.30328772 | 0.04101792 |
| CCDC158 | 1.30172709 | 0.04864399 |
| GSTT2B | 1.29930613 | 0.03187885 |
| OVOL1 | 1.29777311 | 0.04942011 |
| NFASC | 1.2943898 | 0.03109017 |
| C3orf30 | 1.29382815 | 0.04758678 |
| FAM46B | 1.29358791 | 0.04710218 |
| ANGPTL5 | 1.29262477 | 0.03234104 |
| NOVA2 | 1.28987861 | 0.04123225 |
| ERC1 | 1.28902391 | 0.04311467 |
| DPRX | 1.28728737 | 0.03976458 |
| OR52J3 | 1.28533478 | 0.0494609 |
| CFL1 | 1.28179263 | 0.03234104 |
| OR2A7 | 1.27971835 | 0.0428167 |
| MBD3L3 | 1.27744689 | 0.03826527 |
| CAPNS1 | 1.27538093 | 0.0393129 |
| DRP2 | 1.27457547 | 0.03547929 |
| CDK3 | 1.27320599 | 0.04408683 |
| FBXO24 | 1.26694063 | 0.03058902 |
| CD83 | 1.26519927 | 0.03777454 |
| MRGPRX4 | 1.26417594 | 0.03777454 |
| ADAM21 | 1.26406566 | 0.04739298 |
| BSDC1 | 1.26233576 | 0.04123225 |
| MAS1L | 1.26080773 | 0.03777454 |
| CHRNA5 | 1.25837371 | 0.04758678 |
| PITPNM3 | 1.25735779 | 0.04927439 |
| USP10 | 1.25637345 | 0.0379999 |
| TSPAN1 | 1.25450717 | 0.03595259 |
| TNPO2 | 1.25381326 | 0.033227 |
| OSBP2 | 1.2529674 | 0.04728696 |
| AP1M2 | 1.25265971 | 0.03895302 |
| RASSF4 | 1.25244044 | 0.03959362 |
| PALM | 1.25049127 | 0.03234104 |

TABLE 8
Genes conferring sensitivity to Carboplatin (28 uM) in GBM6 cells in the
second round of screening. Genes were identified using the MAGECK algorithm and
filtered to included genes with Beta score (CBP_Diff) of greater than 9 and
adjusted P value (CBP.wald.p.value (SIG)) of less than 0.05 and adjusted P value
(CBP_CTRL.wald.p.valuv (NON-SIG)) of more than 0.05.

| | | CBP_CTRL.wald.p.valuv | |
|---|---|---|---|
| Gene | CBP_Diff | (NON-SIG) | CBP.wald.p.value (SIG) |
| RSPH10B | 10.9473051 | 0.58606 | 0.031425 |
| RCOR1 | 11.8062153 | 0.56984 | 0.032058 |
| AMZ1 | 11.5953221 | 0.52253 | 0.028731 |
| ADRM1 | 9.81074673 | 0.42681 | 0.0068107 |
| GRHPR | 11.0463927 | 0.38052 | 0.022264 |
| UCMA | 10.9100213 | 0.35582 | 0.041893 |
| FNBP1L | 10.3732984 | 0.318 | 0.038487 |
| MRPL52 | 10.9826994 | 0.30748 | 0.0041207 |
| ADAMTS13 | 9.90250008 | 0.29574 | 0.021291 |
| OR2AK2 | 10.8966017 | 0.26956 | 0.016106 |
| BOD1L2 | 9.89063265 | 0.24509 | 0.026905 |
| UCHL5 | 12.6279458 | 0.22356 | 0.01159 |
| RFPL4B | 9.56093227 | 0.22274 | 0.035312 |
| CLPP | 10.2526968 | 0.2097 | 0.037269 |
| ERBB2IP | 10.3544499 | 0.20164 | 0.0078341 |
| NRIP3 | 11.7236369 | 0.19584 | 0.024694 |
| RPL9 | 17.5867373 | 0.19503 | 0.0123 |
| FRAT2 | 12.1034288 | 0.19491 | 0.037227 |
| TDRD5 | 11.9374922 | 0.19329 | 0.011791 |
| CHCHD4 | 9.84010796 | 0.18146 | 0.039168 |
| CNKSR3 | 13.9053211 | 0.15384 | 0.022766 |
| CALM1 | 10.6563762 | 0.15026 | 0.0057965 |
| POLA2 | 11.064722 | 0.1462 | 0.04009 |
| ANP32C | 11.001366 | 0.14381 | 0.039909 |
| ANKEF1 | 9.53811814 | 0.14212 | 0.021878 |
| SYN3 | 9.73779679 | 0.13852 | 0.013269 |
| RAPGEF1 | 13.7098697 | 0.13497 | 0.045266 |
| PTCHD2 | 10.9385573 | 0.13085 | 0.0078398 |
| DEFB107A | 12.4956415 | 0.12777 | 0.0028616 |
| TSPY2 | 10.0258154 | 0.12267 | 0.028712 |
| CAGE1 | 9.57799923 | 0.12181 | 0.021236 |
| EGR4 | 10.6160404 | 0.11664 | 0.028377 |
| HECA | 10.2937855 | 0.11428 | 0.0096346 |
| ABAT | 12.2600634 | 0.11019 | 0.002592 |
| AARS2 | 17.7986406 | 0.10848 | 0.030456 |
| TULP3 | 11.7453453 | 0.103 | 0.029888 |
| TIGD1 | 10.5561224 | 0.098921 | 0.038919 |
| C21orf91 | 10.2990182 | 0.096004 | 0.049328 |
| C6orf132 | 10.3941926 | 0.08985 | 0.0014807 |
| MUL1 | 9.63044606 | 0.08553 | 0.017135 |
| HOXA10 | 9.63042536 | 0.082529 | 0.030795 |
| UBE2I | 12.2749025 | 0.081987 | 0.034013 |
| MCM7 | 13.0833398 | 0.078848 | 0.046864 |
| NUP133 | 11.2310431 | 0.078321 | 0.005114 |
| ZBTB3 | 10.782144 | 0.071838 | 0.0060881 |
| MEX3B | 10.3065308 | 0.071762 | 0.024336 |
| FPGS | 14.6032396 | 0.070853 | 0.021204 |
| OR5H6 | 10.530196 | 0.07037 | 0.031722 |
| CD207 | 11.6422463 | 0.070347 | 0.00071827 |
| CAPRIN1 | 9.57464784 | 0.069403 | 0.048944 |
| CREB3L4 | 10.9846433 | 0.069288 | 0.02208 |
| TST | 9.7215906 | 0.063976 | 0.013125 |
| IL18R1 | 9.78991625 | 0.062205 | 0.021892 |
| ZC3H12C | 13.3099544 | 0.056704 | 0.037445 |
| UVRAG | 10.6229842 | 0.055151 | 0.029005 |
| STX3 | 9.64637219 | 0.054978 | 0.037945 |
| TMEM196 | 11.4583032 | 0.054713 | 0.0048116 |
| P4HA3 | 10.358578 | 0.053639 | 0.0039392 |
| GPKOW | 14.4467535 | 0.052772 | 0.0368 |
| MBTPS2 | 9.5320623 | 0.05181 | 0.039369 |
| LMBR1 | 10.9974683 | 0.050385 | 0.010205 |

TABLE 9
Genes conferring sensitivity to combined treatment of Carboplatin
(28 uM) and Paclitaxel (1 uM) in GBM6 cells in the second round
of screening. Genes were identified using the CB-2 algorithm and filtered
to included genes with log2 fold change of greater than 1.25 and adjusted
P value (FDR_PB) of less than 0.05.

| gene | logFC | fdr_pb |
|---|---|---|
| BAX | 2.06280711 | 0.00314954 |
| CPB2 | 2.04402203 | 0.00765756 |
| API5 | 1.9332759 | 0.00015903 |
| RAB34 | 1.90312828 | 0.00251372 |
| C19orf68 | 1.870548 | 0.00011371 |
| LDLR | 1.85564833 | 0.00302563 |
| LEUTX | 1.83233172 | 9.51E−05 |
| PDE4C | 1.83123884 | 0.00010152 |
| MTRNR2L1 | 1.82149212 | 0.00145469 |
| OR6B1 | 1.8173275 | 0.00013749 |
| NKAIN1 | 1.79706904 | 0.00263547 |
| NDRG4 | 1.79206903 | 5.29E−05 |
| DTX4 | 1.78585904 | 3.65E−05 |
| VWF | 1.7793233 | 3.11E−05 |
| ANXA11 | 1.7732243 | 0.00030174 |
| GJC1 | 1.77249738 | 0.00031808 |
| PID1 | 1.76765142 | 0.00049993 |
| MAS1L | 1.76577006 | 0.0012095 |
| PTGDR2 | 1.75610702 | 0.00020109 |
| ZNF551 | 1.7460609 | 0.00039048 |
| DESI1 | 1.73819015 | 0.00026665 |
| ACTG2 | 1.73350943 | 4.33E−05 |
| ZNF627 | 1.73173808 | 2.50E−05 |
| EPN3 | 1.7310717 | 0.00040524 |
| TLL1 | 1.73033683 | 0.00049644 |
| CRCT1 | 1.72944966 | 5.65E−05 |
| SPOCK3 | 1.71922976 | 0.00021426 |
| KIRREL2 | 1.71670299 | 0.00098382 |
| DEFB124 | 1.71630085 | 0.00010469 |
| NOVA2 | 1.71604005 | 0.00024539 |
| CATSPER3 | 1.71396277 | 0.00057952 |
| SCIMP | 1.71283593 | 0.00142555 |
| OVOL1 | 1.70758954 | 0.0002805 |
| IFNL1 | 1.70665191 | 0.00043302 |
| TRIM61 | 1.700482 | 0.00062386 |
| SLC7A10 | 1.70024149 | 0.00013749 |
| LASP1 | 1.69926684 | 0.00071005 |
| HSD11B2 | 1.69646145 | 0.00014007 |
| TMEM44 | 1.69192435 | 0.00022228 |
| NAB2 | 1.69106771 | 7.89E−05 |
| CYP2A7 | 1.69103075 | 0.00042312 |
| GLRX | 1.6894143 | 0.00077934 |
| CEP97 | 1.67589657 | 0.00027261 |
| UNC79 | 1.67508022 | 5.80E−05 |
| DTD2 | 1.67233092 | 0.0001339 |
| RHOBTB3 | 1.67155794 | 0.00025127 |
| CCL16 | 1.67070136 | 0.00378769 |
| C1orf61 | 1.66910453 | 5.91E−05 |
| TBC1D22A | 1.66483156 | 0.00036771 |
| KCNJ8 | 1.66414377 | 0.00035539 |
| CUBN | 1.66313511 | 6.14E−05 |
| ZNF134 | 1.6609578 | 0.00088453 |
| SOSTDC1 | 1.65748269 | 0.00017945 |
| ITGB4 | 1.65718662 | 2.22E−05 |
| S100B | 1.65683524 | 0.00331428 |
| SLC28A2 | 1.6554742 | 8.71E−06 |
| BVES | 1.65392748 | 0.00115912 |
| ADAP2 | 1.65249778 | 9.11E−05 |
| PODNL1 | 1.65129234 | 0.00134403 |
| HHEX | 1.65018166 | 7.11E−05 |
| CNFN | 1.6494246 | 0.00626681 |
| PKP1 | 1.64935058 | 0.00028336 |
| ARHGAP10 | 1.64871981 | 0.00185446 |
| LPAR2 | 1.64482552 | 0.00013359 |
| KCNJ16 | 1.64095795 | 4.01E−05 |
| PRR29 | 1.64004743 | 0.00074733 |
| KRTAP9-8 | 1.63975126 | 0.00112821 |
| EMD | 1.63964535 | 0.00518406 |
| CASP6 | 1.63707027 | 0.00039605 |
| GJA9 | 1.63587379 | 0.00039042 |
| SCNN1A | 1.63444711 | 0.00012928 |
| PSG3 | 1.63365869 | 0.00094574 |
| APOC2 | 1.63313398 | 0.0008354 |
| DDX11 | 1.63291419 | 0.00081973 |
| GSTO2 | 1.63164647 | 0.00027612 |
| UBE2QL1 | 1.62991313 | 0.00059656 |
| PARP6 | 1.62589858 | 0.00269881 |
| PGPEP1 | 1.62429366 | 7.94E−05 |
| PVRL4 | 1.62226931 | 0.00392314 |
| NAPSA | 1.61846882 | 0.00114097 |
| ZNF846 | 1.61584594 | 0.00020471 |
| OTC | 1.61534492 | 9.95E−05 |
| PKD2L1 | 1.61452902 | 0.00074552 |
| CAPNS1 | 1.61429736 | 4.51E−05 |
| CXCL13 | 1.61294548 | 0.00069899 |
| RAB10 | 1.61257269 | 4.33E−05 |
| TMED7- | 1.61191437 | 0.00011643 |
| TICAM2 | | |
| ALKBH1 | 1.6112246 | 9.34E−05 |
| NTF4 | 1.61117398 | 7.27E−05 |
| RAP1GAP2 | 1.61056345 | 0.00041314 |
| PCDHGB6 | 1.60791585 | 9.11E−05 |
| FAT2 | 1.60753419 | 1.40E−05 |
| BOD1L1 | 1.6048988 | 4.38E−05 |
| SLC20A2 | 1.60366249 | 0.00022681 |
| BCL11A | 1.60323181 | 0.00065611 |
| MIER1 | 1.60292554 | 0.0004773 |
| TIRAP | 1.60241452 | 0.00123198 |

TABLE 10
Genes conferring sensitivity to combined treatment of Carboplatin (28 uM) and
Paclitaxel (1 uM) in GBM6 cells in the second round of screening. Genes were
identified using the MAGECK algorithm and filtered to included genes with
Beta score (CBP_Diff) of greater than 9 and adjusted P value (CBP.wald.p.value
(SIG)) of less than 0.05 and adjusted P value (CBP_CTRL.wald.p.valuv (NON-SIG))
of more than 0.05.

| Gene | COMBO_Diff | COMBO_CTRL.wald.p.value | COMBO.wald.p.value |
|---|---|---|---|
| UBTF | 42.4846564 | 0.13245 | 0.030743 |
| NDUFA11 | 38.998566 | 0.16758 | 0.01903 |
| RPP38 | 36.5360813 | 0.10875 | 0.023734 |
| GOLGA6L10 | 36.1961088 | 0.10311 | 0.020269 |
| MRPL32 | 36.0765922 | 0.089477 | 0.0047858 |
| TDP1 | 34.3977427 | 0.064477 | 0.016753 |
| VAMP8 | 33.9775107 | 0.12302 | 0.039062 |
| STAU2 | 30.5514545 | 0.087514 | 0.044291 |
| NPAT | 30.3606518 | 0.093127 | 0.04967 |
| AADACL2 | 29.8952183 | 0.30557 | 0.035861 |
| HOXA10 | 29.371375 | 0.10448 | 0.0017614 |
| RAB25 | 29.1745246 | 0.06896 | 0.0011332 |
| AKTIP | 29.1244916 | 0.088711 | 0.046915 |
| PPIA | 29.0485552 | 0.16591 | 0.013128 |
| RUFY1 | 28.9283269 | 0.094818 | 0.023912 |
| C9orf41 | 28.8521802 | 0.19723 | 0.049724 |
| ANKLE2 | 28.7191774 | 0.17021 | 0.033613 |
| F2RL1 | 28.41402 | 0.079155 | 0.025308 |
| FLI1 | 28.1222733 | 0.090746 | 0.028647 |
| NLRP6 | 27.8145073 | 0.23956 | 0.010864 |
| UBE2C | 27.6381992 | 0.43264 | 0.037179 |
| ADAMTS13 | 26.8944567 | 0.061774 | 0.00085282 |
| SLC6A11 | 26.8617629 | 0.13498 | 0.016425 |
| B3GNT5 | 26.7435159 | 0.057008 | 0.0088104 |
| CYP2A6 | 26.2638482 | 0.10394 | 0.01956 |
| MAFF | 26.0587194 | 0.067987 | 0.011689 |
| KCNS2 | 25.9451095 | 0.21555 | 0.020252 |
| CREB3L4 | 25.5679531 | 0.21626 | 0.022319 |
| EBLN1 | 25.3002399 | 0.081597 | 0.0023017 |
| SPATA20 | 25.2050423 | 0.11756 | 0.018368 |
| SAMD4B | 25.111225 | 0.13156 | 0.045285 |
| TM9SF3 | 25.0104226 | 0.19473 | 0.017823 |
| TP53I3 | 24.8434904 | 0.23851 | 0.041948 |
| TAGLN | 24.4963291 | 0.091582 | 0.017816 |
| UQCC3 | 24.3582873 | 0.089161 | 0.0014323 |
| LENEP | 24.0767592 | 0.18945 | 0.034398 |
| PCDH12 | 23.9539532 | 0.072749 | 0.039202 |
| CPSF3 | 23.7034381 | 0.13172 | 0.0053305 |
| C1QTNF7 | 23.6015542 | 0.055678 | 0.0072854 |
| LSM2 | 23.4888551 | 0.077482 | 0.017155 |
| NLRP13 | 23.1023487 | 0.36605 | 0.045578 |
| NTS | 22.9679827 | 0.16619 | 0.0061692 |
| DUSP2 | 22.8176936 | 0.11858 | 0.0032233 |
| BPIFA2 | 22.6323799 | 0.22216 | 0.036319 |
| MYO1C | 22.5919028 | 0.054024 | 0.028485 |
| UNC13A | 22.3882484 | 0.074907 | 0.040818 |
| ENPP1 | 22.3638768 | 0.056956 | 0.041645 |
| CATSPERG | 22.2953734 | 0.063537 | 0.022894 |
| SAR1A | 22.0301103 | 0.22712 | 0.033963 |
| ZNF207 | 21.9912457 | 0.058133 | 0.044375 |
| DSG1 | 21.9520076 | 0.079833 | 0.0498 |
| DMTN | 21.8121749 | 0.071365 | 0.020611 |
| HCFC1R1 | 21.6097303 | 0.20842 | 0.017553 |
| RWDD2A | 21.5756046 | 0.059982 | 0.032079 |
| ARHGAP10 | 21.5517296 | 0.50423 | 0.020987 |
| FBXO2 | 21.511577 | 0.19833 | 0.030184 |
| DYM | 21.4785804 | 0.26227 | 0.014459 |
| MPZL1 | 21.3435681 | 0.1826 | 0.042608 |
| C8orf4 | 21.183847 | 0.070534 | 0.0012136 |
| ACRV1 | 21.1209913 | 0.35968 | 0.013845 |
| PTPMT1 | 21.0591217 | 0.1186 | 0.021766 |
| FANCE | 21.0493406 | 0.13138 | 0.00099359 |
| FBXO5 | 20.9875243 | 0.057166 | 0.0046649 |
| DRG2 | 20.9486332 | 0.052756 | 0.020328 |
| DAZAP1 | 20.9467051 | 0.22801 | 0.004018 |
| GATA5 | 20.8873749 | 0.15152 | 0.012183 |
| BACH2 | 20.7527567 | 0.18219 | 0.0058837 |
| API5 | 20.699427 | 0.29962 | 0.0026545 |
| KIAA0232 | 20.6790017 | 0.059457 | 0.0066552 |
| PSME3 | 20.6442359 | 0.14319 | 0.043094 |
| CLUH | 20.4501375 | 0.17989 | 0.04861 |
| GPR137C | 20.4176266 | 0.053176 | 0.013213 |
| PHYKPL | 20.4080549 | 0.20654 | 0.01246 |
| SOX15 | 20.2451377 | 0.21217 | 0.019054 |
| FBXO28 | 20.2348867 | 0.09152 | 0.0089653 |
| VWC2L | 20.2305623 | 0.2502 | 0.010653 |
| PGGT1B | 20.1565523 | 0.51906 | 0.047228 |
| SERPINB13 | 20.1531665 | 0.21534 | 0.0073561 |
| RUNX1 | 19.991503 | 0.19037 | 0.035871 |
| KIAA1755 | 19.9226201 | 0.061522 | 0.0067583 |
| NRSN2 | 19.892138 | 0.087854 | 0.0075055 |
| RDH12 | 19.8501867 | 0.27284 | 0.048868 |
| TNFAIP2 | 19.846211 | 0.079514 | 0.0040255 |
| PCDHGA2 | 19.7938744 | 0.14764 | 0.011094 |
| IGF2 | 19.700762 | 0.058761 | 0.022459 |
| NDRG1 | 19.6097517 | 0.15591 | 0.04254 |
| RFT1 | 19.5955057 | 0.38649 | 0.016277 |
| PDE12 | 19.5450057 | 0.053784 | 0.010508 |
| ANOS1 | 19.5341419 | 0.10674 | 0.0077716 |
| UBE2G2 | 19.4757175 | 0.32798 | 0.0090158 |
| CKM | 19.3854725 | 0.075769 | 0.025383 |
| FANCC | 19.3166741 | 0.34631 | 0.0091791 |
| NEUROG2 | 19.2232198 | 0.49931 | 0.016222 |
| PSME1 | 19.1714804 | 0.072982 | 0.036032 |
| ATXN3L | 19.1433369 | 0.2442 | 0.044492 |
| OVCA2 | 19.0698037 | 0.053239 | 0.040985 |
| RASA4B | 19.0476017 | 0.08866 | 0.02606 |
| MMEL1 | 18.996168 | 0.094242 | 0.046825 |
| CLEC4M | 18.9857914 | 0.068114 | 0.0060842 |
| RFESD | 18.880966 | 0.093859 | 0.019353 |

### Equivalents

Unless otherwise defined, all technical and scientific terms used herein have the same meaning as commonly understood by one of ordinary skill in the art to which this technology belongs.

Although the foregoing refers to particular preferred embodiments, it will be understood that the present invention is not so limited. It will occur to those of ordinary skill in the art that various modifications may be made to the disclosed embodiments and that such modifications are intended to be within the scope of the present invention.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, GenBank citations, ATCC citations, and other references mentioned herein are expressly incorporated by reference in their entirety, to the same extent as if each were incorporated by reference individually. In case of conflict, the specification, including definitions, will control.

What is claimed is:

1. A method for cancer biomarker gene identification, comprising:
a) obtaining genome-wide CRISPR screen data for cancer cells exposed to at least one chemotherapeutic agent;
b) identifying candidate genes from the CRISPR screen;
c) performing DNA methylation analysis of probes associated with the genes in (b) in cancer samples collected from a subject with the cancer and treated with the at least one chemotherapeutic agent to determine DNA methylation value of the probes;
d) quantifying a cancer-progression metric of interest in the subject; and
e) correlating the DNA methylation values of (c) with the cancer-progression metric of interest in (d),
wherein the candidate genes are associated with susceptibility to the at least one chemotherapeutic agent, wherein methylation of at least one probe and cancer-progression metric are correlated in biomarker genes, wherein the probe methylation value indicates cancer susceptibility to the chemotherapeutic agent, and wherein the correlation can be positive or negative, dependent on probe and associated gene, and the cancer-progression metric of interest.

2. The method of claim 1, wherein the cancer is selected from breast cancer, ovarian cancer, Kaposi's sarcoma, or glioma, optionally wherein the glioma is glioblastoma.

3. The method of claim 1, wherein the chemotherapeutic agent is selected from bleomycin, cyclophosphamide, doxorubicin, epirubicin, etoposide, 5-fluorouracil, methotrexate, oxaliplatin, temozolomide, cisplatin, paclitaxel and/or carboplatin.

4. The method of claim 1, wherein the CRISPR screen is performed with the Brunello sgRNA library.

5. The method of claim 1, wherein the cancer cells are glioma cells, optionally wherein the glioma is glioblastoma.

6. The method of claim 1, wherein the cancer-progression metric of interest is subject survival or tumor growth.

7. A cancer biomarker gene determined using the method of claim 1, wherein the biomarker gene is a gene identified as contributing susceptibility to a chemotherapeutic agent, optionally wherein the chemotherapeutic agent is selected from paclitaxel and carboplatin.

8. The biomarker gene of claim 7, wherein the biomarker gene is selected from IL1R1, GRIN3B, ZBTB20, EME2, TNRC6C, FLT3, TMEM131, and TEX22.

9. The biomarker gene of claim 7, wherein the biomarker gene is a gene which contributes susceptibility to two chemotherapeutic agents, optionally wherein the two chemotherapeutic agents are paclitaxel and carboplatin.

10. The biomarker gene of claim 7, wherein the biomarker gene contributes susceptibility to at least three chemotherapeutic agents.

11. A method of treatment of cancer in a subject comprising:
a) quantifying DNA methylation of a probe correlated with the cancer biomarker gene of claim 7 in a cancer sample from a subject, wherein the sample is exposed to a chemotherapeutic agent prior to methylation analysis;
b) identifying the cancer as susceptible to the chemotherapeutic agent based on the methylation level; and
c) administering to the subject the chemotherapeutic agent that the cancer is susceptible to,
wherein the methylation level of the probe is of a value correlated with susceptibility to the chemotherapeutic agent.

12. The method of claim 11, wherein the cancer is selected from breast cancer, ovarian cancer, Kaposi's sarcoma, or glioblastoma.

13. The method of claim 11, wherein the chemotherapeutic agent is selected from bleomycin, cyclophosphamide, doxorubicin, epirubicin, etoposide, 5-fluorouracil, methotrexate, oxaliplatin, temozolomide, cisplatin, paclitaxel and/or carboplatin.

14. The method of claim 11, wherein the subject is a human or other mammal.

15. A method of identifying a cancer in a subject that is susceptible to a chemotherapeutic agent comprising:
a) quantifying methylation levels of a probe correlated with a cancer biomarker gene of claim 1 in a cancer sample from a subject, wherein the sample is exposed to the chemotherapeutic agent prior to methylation analysis; and
b) identifying the cancer as susceptible to treatment with the chemotherapeutic agent based on the methylation level,
wherein the cancer biomarker gene is identified as contributing susceptibility to the chemotherapeutic agent, and
wherein the methylation level of the probe is of a value correlated with susceptibility to the chemotherapeutic agent.

16. The method of claim 15, wherein the cancer is selected from breast cancer, ovarian cancer, Kaposi's sarcoma, or glioblastoma.

17. The method of claim 15, wherein the chemotherapeutic agent is selected from bleomycin, cyclophosphamide, doxorubicin, epirubicin, etoposide, 5-fluorouracil, methotrexate, oxaliplatin, temozolomide, cisplatin, paclitaxel and/or carboplatin.

18. The method of claim 15, wherein the subject is a human or other mammal.

19. The method of claim 1, wherein the CRISPR screen is a pooled CRISPR screen.

20. The method of claim 1, wherein the CRISPR screen is an arrayed CRISPR screen.

21. The method of claim 11, wherein the cancer is glioblastoma.

22. The method of claim 1, wherein the methylation of at the least one probe and cancer-progression metric in biomarker genes are linearly correlated.
